(19) United States
(12) Patent Application Publication
(10) Pub. No.: US 2018/0362660 A1
(43) Pub. Date: Dec. 20, 2018

(54) Anti-penumbra monoclonal antibodies for detection and therapy of normal and abnormal B lymphocytes

(71) Applicant: Schickwann Tsai, Salt Lake City, UT (US)

(72) Inventor: Schickwann Tsai, Salt Lake City, UT (US)

(21) Appl. No.: 15/436,855

(22) Filed: Feb. 19, 2017

Related U.S. Application Data
(60) Provisional application No. 62/391,749, filed on May 9, 2016.

(51) Int. Cl. C07K 16/36; C07K 16/30

(52) U.S. Cl. CPC C07K 16/36; C07K 16/3061; C07K 2317/94; C07K 2317/31; C07K 2317/565; C07K 2317/33

(57) ABSTRACT

Penumbra is the newest member of the tetraspanin superfamily of membrane proteins. A major obstacle in penumbra research has been the lack of monoclonal antibodies against the native penumbra. In this invention, we detail the establishment and characterization of monoclonal antibodies that recognize both human and mouse penumbras on living cells. Furthermore, we created chimeric mouse-human IgG1 antibodies from these mouse monoclonal antibodies. Using these antibodies, we demonstrate for the first time that penumbra is expressed on the surface of virtually all CD19+ or CD20+ B lymphocytes in blood, bone marrow, spleen and lymph nodes. In vivo, these monoclonal antibodies shrank lymphoid follicles in spleen. Thus, these antibodies establish penumbra as a novel B cell marker with a wider range of expression level than CD19 or CD20. These monoclonal antibodies pave the way for new research and potential therapeutic applications in immunology, hematology and oncology.

### CROSS REFERENCE TO RELATED APPLICATIONS

This application claims benefit of priority of U.S. Provisional Patent Application Ser. No. 62/391,749 filed May 9, 2016 by the present inventor, the disclosure of which is hereby incorporated by reference in its entirety.

### SEQUENCE LISTING

A text file named “PenumbraMoAbSeq_ST25” with a total of 6K bytes and created on Feb. 17, 2017 using PatentIn35 is submitted via PTO EFS as part of this application.

### BACKGROUND OF THE INVENTION

Penumbra, abbreviated as “Pen” (the gene) or “pen” (the protein), was first cloned as a gene differentially expressed in erythroblasts by the present inventor in 1998 and further characterized and reported in 2000 (ref. 1), 2005 (ref. 2) and 2007 (ref. 3).

Pen is the newest member of the tetraspanin superfamily and is designated as tetraspanin 33, abbreviated as Tspan33, in GenBank. It is also a member of the recently recognized “eight-cysteine tetraspanin” subfamily (TspanC8)(ref. 4-6). It was envisaged at the time of the cloning of Pen that anti-pen monoclonal antibodies (mAbs) could be important tools in hematology and immunology since many widely used blood cell markers such as CD9, CD37, CD53, CD63, CD81, CD82 and CD151 were all tetraspanins while CD20 resembles tetraspanins in size and the overall domain organization (infra).

Tetraspanins are evolutionarily conserved transmembrane proteins (ref. 7-9). There are thirty-three tetraspanins in both mouse and human. All tetraspanins contain short intracellular amino- and carboxyl-termini, four transmembrane domains, two extracellular domains (ECD1 and ECD2) and conserved amino acids at key positions (ref. 10). The ECD2 of tetraspanin contains four, six or eight cysteines, allowing the formation of two, three or four disulfide bonds that play crucial roles in creating a special fold in the large ECD2. Some tetraspanins can self-associate laterally to form large “tetraspanin-enriched microdomains” (ref. 11-13). Several tetraspanins have been shown to interact with specific membrane proteins, usually via ECD2, and incorporate them into “tetraspanin-enriched microdomains” and thereby modulate the efficiency of signal transduction by specific membrane proteins (ref. 14).

The membrane protein partners of tetraspanins are diverse and include CD19 (associates with tetraspanin 28, a.k.a. CD81 and tetraspanin 27, a.k.a. CD82)(ref. 15-20), integrin α₄β₁ (associates with tetraspanin 28, tetraspanin 27, tetraspanin 30, a.k.a. CD63, and tetraspanin 25, a.k.a. CD53)(ref. 21), integrins α₃β₁ and α₆β₁ (associate with tetraspanin 24, a.k.a. CD151)(ref. 22), P-selectin (associates with tetraspanin 30, a.k.a. CD63)(ref. 23), and vascular cell adhesion molecule-1 or VCAM-1 (associates with tetraspanin 24)(ref. 24), to name just a few.

Other membrane proteins that also contain four transmembrane domains and two extracellular domains but not the conserved amino acids at key positions are not included in the tetraspanin superfamily. A notable example is the B cell marker CD20 (ref. 25), which is a member of the CD20/FcεRIβ superfamily. Although CD20 resembles tetraspanins in size (297 amino acids vs. 283 amino acids in pen) and domain organization (short intracellular amino- and carboxyl-termini, four transmembrane domains, one small ECD1 between the first and second transmembrane domains and one large ECD2 between the third and fourth transmembrane domains), it shares no conserved amino acids at key positions with tetraspanins and has only one disulfide bond in ECD2 (ref. 26, 27). Therefore, CD20 is not classified as a tetraspanin. It appears that the predecessors of the tetraspanin and the CD20/FcεRIβ superfamilies evolved separately soon after their debut. Of note, CD20 is the target of the highly effective therapeutic mAb, RITUXIMAB™, for non-Hodgkin's B cell lymphomas, chronic B cell leukemia and autoimmune disorders (ref. 27-29).

Very little is known about TspanC8 except that they all have eight cysteines in their ECD2 and that most TspanC8, including pen, can form a specific complex with the membrane protein “A Disintegrin And Metalloprotease 10” (ADAM10)(ref. 5, 6, 30). TspanC8 is required for the biosynthesis, maturation and trafficking of ADAM10 from endoplasmic reticulum to the Golgi apparatus and finally to the cell surface or other membrane compartments. In the absence of TspanC8, ADAM10 remains trapped in the endoplasmic reticulum after synthesis (ref. 5, 6, 30). This effect is quite dramatic visually (ref. 6). An interesting parallel is seen in CD19, another quasi-pan B cell marker beside CD20 and a component of B Cell Receptor or BCR complex (ref. 15-17, 20). Like ADAM10, CD19 also remains trapped in the endoplasmic reticulum in the absence of its companion, tetraspanin 28, a.k.a. CD81 (ref. 31).

The mouse and human pens are 97.2% identical in amino acid sequence and 98.3% identical in ECD2 (ref. 3). For comparison, the human β-globin and mouse β1-globin share only 80.3% identity in amino acid sequence and the human and mouse CD20 are only 73% identical in amino acid sequence. The near identity (97.2%) of mouse and human pens indicates that there is an exceptionally high evolutionary pressure against their sequence variation, more so than for β-globin and CD20.

A major obstacle in the study of pen and TspanC8 in general has been the lack of mAbs against these proteins, especially the native forms on living cells (ref. 5; p. 39763, line 41). The near identity of mouse and human pen amino acid sequences may be part to blame in the case of hpen, but the difficulty in recreating the native topology of TspanC8 in vitro sans cell membrane is probably the main reason. Although a few polyclonal antibody preparations became available briefly (e.g. Abcam, cat. no. ab87543; Santa Cruz, cat. no. sc-138518; Atlas Antibodies HPA020357), all were raised against synthetic peptides corresponding to the intracellular, carboxyl terminus or part of ECD2 of hpen and did not recognize the native hpen protein or the native ECD2 on living cells. There was also the issue of off-targets with polyclonal antibodies raised against synthetic peptides. Therefore, their utility was limited. Our own efforts at creating anti-Pen mAbs began well before 2013 and underwent many changes in protocols.

Due to the lack of antibodies with proven specificity for native pen, most studies so far have relied on Northern or Western analysis or quantitative reverse transcriptase-polymerase chain reaction (qRT-PCR)(ref. 3, 32). These studies revealed that Pen has a restricted expression pattern with most Pen mRNA found in bone marrow, spleen and kidney (ref. 3 and unpublished human data, present inventor). This contrasts with the ubiquitous or quasi-ubiquitous expression pattern of most other tetraspanins such as CD81 (ref. 8, 9). The detection of Pen mRNA and protein in bone marrow was initially thought to reflect their expression in erythroblasts (ref. 3). The expression of Pen mRNA in mouse spleen was also initially thought to be attributable to erythroblasts as there is significant extramedullary erythropoiesis in the spleens of adult mice. However, the level of PEN mRNA in human spleen is also very high despite the fact that there is no extramedullary erythropoiesis in normal adult human spleens (ref. 3). This apparent contradiction indicates that an additional cell type(s) in the spleen may express mouse Pen or human PEN mRNA. In this regard, recent DNA array data, RT-PCR and Western blot analyses indicated that in vitro activated (by anti-CD40 mAb plus IL-4, or CD40 ligand plus IL-4, or CpG plus pokeweed mitogen/PWM plus pansorbin) mature yet naive human B cells (i.e. fully differentiated B cells with functional B Cell Receptor but have not encountered the corresponding antigens yet) upregulated human PEN mRNA compared with resting B cells (ref. 32). A similar pattern of mouse Pen expression was noted in resting and in vitro activated (by lipopolysaccharide/LPS plus IL-4) mouse D cells (ibici).

In this application, we describe the establishment of mAbs as exemplified by 29A6.2, 41B10.13 and 59E6.8 and their chimeric mouse-human IgG1 versions mh29A6.2, mh41B10.13 and mh59E6.8, which secret mAbs recognizing both mouse and human pen proteins (abbreviated as mpen and hpen according to the protein naming convention) in living cells specifically. Using these mAbs we are able to demonstrate for the first time that pen is expressed on the surface of virtually all CD19+ or CD20+ or B220+ B cells regardless of their activation status in all primary and secondarily lymphoid tissues examined as well as in peripheral blood. However, its expression profile within the B lymphocyte population is very different from that of CD19 or CD20 in that it has a very wide range of expression level. In addition, these mAbs revealed that pen is also expressed in a very small subpopulation of erythrocytes and/or erythroblasts in bone marrow and a subset of newly released erythrocytes in peripheral blood, consistent with the previous report (ref. 3). Furthermore, anti-pen mAbs dramatically reduced the number and size of lymphoid follicles in the spleen and shrank the size of the spleen by about 50% when they were administered in vivo for 5-7 days. Thus these anti-pen mAbs enabled us to establish pen as a new cell surface marker of human and mouse B lymphocytes and provide new tools for research, diagnosis, prognostication and therapy in immunology, hematology and oncology.

### BRIEF SUMMARY OF THE INVENTION

This Invention relates to the creation of anti-penumbra (tetraspanin 33; mouse and human) mAbs and the surprise findings that they recognize virtually all human and mouse B lymphocytes and that pen has a wider range of expression level on B lymphocytes than other B cell markers such as CD19 and CD20.

In one embodiment, the invention provides an IgM mAb “29A6.2” that binds to both human and mouse pen protein on living cells: a heavy chain with the complete variable region amino acid sequence (including signal peptide) corresponding to SEQ ID NO:1 and a kappa light chain with the complete variable region amino acid sequence (including signal peptide) corresponding to SEQ ID NO:4.

In another embodiment, the invention provides an IgM mAb “41B10.13” that binds to both human and mouse pen protein on living cells: a heavy chain with the complete variable region amino acid sequence (including signal peptide) corresponding to SEQ ID NO:2 and a kappa light chain with the complete variable region amino acid sequence (including signal peptide) corresponding to SEQ ID NO:4.

In an additional embodiment, the invention provides an IgM mAb “59E6.8” that binds to both human and mouse pen protein on living cells: a heavy chain with the complete variable region amino acid sequence (including signal peptide) corresponding to SEQ ID NO:3 and a kappa light chain with the complete variable region amino acid sequence (including signal peptide) corresponding to SEQ ID NO:4.

In still another embodiment, the invention provides chimeric mouse-human IgG1 mAbs “mh29A6.2”, “mh41B10.13”, and “mh59E6.8”, in which the variable regions of heavy and light chains of mAbs 29A6.2, 41B10.13 and 59E6.8 have been grafted in frame onto the constant domains of human IgG1 heavy chain and kappa light chain. These chimeric antibodies retain the pen-binding specificity of mouse mAbs but adopt the structures, properties and functions of the constant regions of human IgG1 and kappa light chain and greatly increase their potential as diagnostic tools and immunotherapy drugs in humans.

### BRIEF DESCRIPTION OF THE DRAWINGS

FIGS. 1A and 1B show that mAb 29A6.2 recognizes cell-surface pen specifically. Cell culture supernatant (300 microliter per 1×10⁵ target cells) of hybridoma 29A6.2 was incubated for 40 min. with BaF3/mpen that had been pretreated with FcBlock to prevent binding of mouse immunoglobulins via Fc receptors. Bound mAbs were detected by GAMIgs-FITC and analyzed by flow cytometry. FIG. 1A shows the histogram of BaF3/mpen incubated with the culture supernatant of parental SP2/0 AG14 (negative control). FIG. 1B shows the histogram of BaF3/mpen exposed to the culture supernatant of hybridoma 29A6.2. Horizontal axis represents the intensity of FITC fluorescence.

FIGS. 2A to 2F are indirect immunofluorescence micrographs of living cells stained with mAb 29A6.2. Cell culture supernatant (300 microliter per 1×10⁵ target cells) of hybridoma 29A6.2 was incubated with BaF3/pcDNA3.1 vs. BaF3/mpen or BaF3/hpen. Bound antibodies were detected by GAMIgs-FITC or GAMIgs-PE. FIG. 2A is a phase-contrast micrograph of BaF3/pcDNA3.1 (negative control) stained with 29A6.2 supernatant. FIG. 2B is an immunofluorescence micrograph of the same cells shown in 2A. FIG. 2C is a phase-contrast micrograph of BaF3/mpen stained with 29A6.2 supernatant. FIG. 2D is an immunofluorescence micrograph of the same cells shown in 2C. Cells with fuzzy appearance were above or below the focal plane. FIG. 2E is a phase-contrast micrograph of BaF3/hpen (i.e. human pen) stained with 29A6.2 supernatant. FIG. 2F is an immunofluorescence micrograph of the same cells shown in 2E. (The capping effect seen in FIG. 2F is due to warming of specimen.) Bars indicate 200 micron. mAbs 41B10.13 and 59E6.8 yielded similar findings (not shown).

FIGS. 3A to 3P demonstrate that anti-pen mAbs specifically recognize splenic B cells that are CD19+ or CD20+ or B220 (CD45R)+. Balb/C mouse splenic cells were lysed with ACK lysis buffer, treated with FcBlock and stained with PE-conjugated lineage-specific mAbs plus isotype-FITC (all left-hand panels) or 29A6.2-FITC (all right-hand panels). Flow cytometry data were analyzed using FloJo v.9 software. FIG. 3A: Isotype-PE vs. isotype-FITC. Thin gray lines delineate modified quadrants. Numbers in various modified quadrants indicate %. FIG. 3B: Isotype-PE vs. 29A6.2-FITC. FIG. 3C: Anti-CD19-PE vs. isotype-FITC. FIG. 3D: Anti-CD19-PE vs. 29A6.2-FITC. The entire population of CD19+ cells was recognized by mAb 29A6.2-FITC. FIG. 3E: Anti-B220-PE vs. isotype-FITC. B220+ cells segregated into B220^{Hi} and B220^{Lo} populations. FIG. 3F: Anti-B220-PE vs. 29A6.2-FITC. Virtually all B220^{Hi} and B220^{Lo} cells were recognized by 29A6.2-FITC. FIG. 3G: Anti-CD20-PE vs. isotype-FITC. FIG. 3H: Anti-CD20-PE vs. 29A6.2-FITC. FIG. 3I: Anti-CD3-PE vs. isotype-FITC. FIG. 3J: Anti-CD3-PE vs. 29A6.2-FITC. FIG. 3K: TER119-PE vs. isotype-FITC. FIG. 3L: TER119-PE plus 29A6.2-FITC. FIG. 3M: Anti-Gr-1-PE vs. isotype-FITC. The Gr-1^{Lo} cells were false positives due to excess antibody. FIG. 3N: Anti-Gr-1-PE plus 29A6.2-FITC. FIG. 3O: Anti-Mac-1-PE vs. isotype-FITC. FIG. 3P: Anti-Mac-1-PE vs. 29A6.2-FITC. Data shown were lymphocyte-gated; however, the ungated live cells yielded essentially the same findings (not shown). Very similar staining patterns were obtained using 41B10.13-FITC or 59E6.8-FITC (not shown). Staining with anti-CD14-PE or DX5-PE was negative. These results indicate that anti-pen mAbs recognized virtually all CD19+ or B220+ or CD20+ splenic B lymphocytes as well as a very small population of TER119 (glycophorin A)^{Lo} erythrocytes and/or erythroblasts in spleen. The overwhelming majority of mature erythrocytes (i.e. before ACK lysis) did not stain with anti-pen mAbs (not shown).

FIGS. 4A to 4I reveal that anti-pen mAbs recognize splenic B lymphocytes regardless of their activation status. Balb/C mouse splenocytes were analyzed by flow cytometry before and after stimulation with LPS (10 μg/ml) and mIL-4 (10 μg/ml) for 24-72 hrs. Cells were stained with anti-CD19-PE, 41B10.13-FITC and anti-CD69-APC or anti-CD86-APC simultaneously. CD69 and CD86 are commonly used markers of B cell activation. Flow cytometry data were analyzed using FloJo v.10 software. FIG. 4A: Anti-CD19-PE vs. isotype-FITC dot plot on day 0. FIG. 4B: Anti-CD19-PE vs. 41B10.13-FITC dot plot on day 0. Most CD19+ splenocytes showed some staining with anti-pen mAb. FIG. 4C: Anti-CD19-PE vs. 41B10.13-FITC dot plot after 72-hr. stimulation with LPS and mIL-4. Please note that there was no significant increase in the expression of pen after activation. Similar results were obtained after stimulation with LPS and mIL-4 for 24 or 48 hrs. (not shown). All CD19+ cells in 4A, 4B and 4C were gated and analyzed for CD69 and CD86 expression and the results are shown in 4D to 4F (for CD69 expression) and 4G to 4I (for CD86 expression). FIG. 4D: Histogram of isotype-APC staining (negative control) on day 0. FIG. 4E: Histogram of anti-CD69-APC staining on day 0. FIG. 4F: Histogram of anti-CD69-APC staining 72 hrs. after stimulation with LPS and mIL-4. FIG. 4G: Histogram of Isotype-APC staining (negative control) on day 0. FIG. 4H: Histogram of anti-CD86-APC staining on day 0. FIG. 4I: Histogram of anti-CD86-APC staining after 72-hr. stimulation with LPS and mIL-4. The increased CD69 and CD86 (and CD80, not shown) expression in 4F and 4I confirmed the activation of B lymphocytes by LPS+mIL4. Similar data were obtained after 24- or 48-hr. stimulation (not shown).

FIGS. 5A to 5P show that anti-pen mAbs also recognize bone marrow B lymphocytes. Balb/C mouse bone marrow cells were lysed with ACK lysis buffer, treated with FcBlock and stained with PE-conjugated lineage-specific mAbs plus isotype-FITC (all left-hand panels) or 29A6.2-FITC (all right-hand panels). Flow cytometry data were analyzed using FloJo v.10 software. The number in each quadrant denotes %. FIG. 5A: A dot plot of isotype-PE vs. isotype-FITC. FIG. 5B: A dot plot of isotype-PE vs. A296.2-FITC. FIG. 5C: Anti-CD19-PE vs. isotype-FITC. FIG. 5D: Anti-CD19-PE vs. 29A6.2-FITC. FIG. 5E: Anti-B220-PE vs. isotype-FITC. FIG. 5F: Anti-B220-PE vs. 29A6.2-FITC. Please note that B220^{Hi} cells are also Pen^{Hi}. FIG. 5G: Anti-CD20-PE vs. isotype-FITC. FIG. 5H: Anti-CD20-PE vs. 29A6.2-FITC. FIG. 5I: Anti-CD138-PE vs. isotype-FITC. FIG. 5J: Anti-CD138-PE vs. 29A6.2-FITC. Please note that most CD138+ cells do not express pen or express very low levels of pen. FIG. 5K: Anti-CD117-PE vs. isotype-FITC. FIG. 5L: Anti-CD117-PE vs. 29A6.2-FITC. FIG. 5M: TER119-PE vs. isotype-FITC. Please note bone marrow TER119+ erythrocytes and/or erythroblasts exhibit high autofluorescence. FIG. 5N: TER119-PE vs. 29A6.2-FITC. FIG. 5O: Anti-Mac-1-PE vs. isotype-FITC. FIG. 5P: Anti-Mac-1-PE vs. 29A6.2-FITC. Please note that some Mac-1+(=CD11b+) cells also express low levels of pen. These are likely B cells expressing integrin αM chain (CD11b). Data shown were lymphocyte-gated. However, the ungated live cells yielded similar findings (not shown). Very similar results were obtained using 41B10.13-FITC or 59E6.8-FITC (not shown). Staining with anti-CD14-PE or DX5-PE was negative. These results indicate that anti-pen mAbs recognize the majority of CD19+ or B220+ or CD20+ B cells as well as a small population of glycophorin A⁺ erythrocytes and/or erythroblasts in bone marrow.

FIGS. 6A to 6D confirm that anti-pen mAbs also recognize CD19+ and CD20+ B cells in peripheral blood. Balb/C mouse peripheral blood cells were lysed by ACK buffer, treated with FcBlock and then stained with various flurochrome-conjugated antibodies. FIG. 6A: A dot plot of anti-CD19-PE vs. isotype-FITC staining. FIG. 6B: A dot plot of anti-CD19-PE vs. 29A6.2-FITC staining. Virtually all peripheral blood CD19+ B cells expressed medium to high levels of cell surface pen. FIG. 6C: A dot plot of anti-CD20-PE vs. isotype-FITC staining. FIG. 6D: A dot plot of anti-CD20-PE vs. 29A6.2-FTIC staining. Virtually all CD20+ B cells in blood also expressed pen. Similar findings were obtained using 41B10.13-FITC or 59E6.8-FITC (not shown). Co-staining with other lineage-specific mAbs including anti-CD3, -CD4, -CD8, -CD14, Gr-1, Mac-1 or DX5 was essentially negative (not shown).

FIGS. 7A to 7F illustrate that mouse lymph node B cells also express pen. Single cell preparations of Balb/C mouse cervical and omental lymph nodes were lysed with ACK buffer, treated with FcBlock and stained with various fluorochrome-conjugated antibodies. FIG. 7A: A dot plot of anti-CD19-PE vs. isotype-FITC staining. FIG. 7B: A dot plot of anti-CD19-PE vs. 29A6.2-FITC staining. Note virtually all CD19+ B cells also expressed pen. FIG. 7C: A dot plot of anti-CD20-PE vs. isotype-FITC staining. FIG. 7D: A dot plot of anti-CD20-PE vs. 29A6.2-FITC staining. Virtually all CD20+ B cells expressed some levels of pen. FIG. 7E: A dot plot of anti-CD138-PE vs. isotype-FITC staining. FIG. 7F: A dot plot of anti-CD138-PE vs. 29A6.2-FITC staining. Some CD138+ cells (usually plasma cells) exhibit low levels of pen expression. Co-staining with other lineage-specific mAbs including anti-CD4, -CD8, -CD14, Gr-1 or DX5 was negative (not shown).

FIGS. 8A to 8D illustrate that anti-mpen mAbs can also recognize human peripheral blood B lymphocytes. Human blood MNCs were prepared by a step gradient of Ficoll-Paque Plus, followed by staining with PE-conjugated lineage-specific antibodies and anti-mpen mAb 29A6.2-FITC. FIG. 8A: A dot plot of anti-hCD19-PE vs. isotype-FITC. FIG. 8B: A dot plot of anti-hCD19-PE vs. 29A6.2-FITC. Note virtually all CD19+ human B cells exhibited some level of cell surface pen. FIG. 8U: A dot plot of anti-hCD235a (glycophorin A)-PE vs. isotype-FITC. Please note that the overwhelming majority (>99.9999%) of mature erythrocytes (before removal by ACK buffer lysis) exhibited levels of anti-hCD235a staining that were out of the range of the plots shown here. They had no detectable staining with mAb 29A6.2. FIG. 8D: A dot plot of anti-hCD235a-PE vs. 29A6.2-FITC. Some hCD235a-PE^{Lo} erythrocytes showed very low levels of hpen expression. These hCD235a-PE^{Lo} hpen^{Lo} cells most likely represented a subset of young erythrocytes that had survived ACK lysis buffer treatment. Staining using antibodies against other major hematopoietic lineages (including anti-hCD3, -hCD4, -hCD8 and -hCD14) were negative (not shown).

FIGS. 9A to 9F show that expression of hpen in human B lymphocytes is not increased after activation. Low density (ρ<1.077 g/dL) human peripheral blood MNCs were analyzed on day 0 and after stimulation with hCD40L (200 ng/ml) plus hIL-4 (20 ng/ml) for 3 days. FIG. 9A: A dot plot of anti-hCD19-PE vs. isotype-FITC staining on day 0. FIG. 9B: A dot plot of anti-hCD19-PE vs. 41B10.13-FITC staining on day 0. FIG. 9C: A dot plot of anti-hCD19-PE vs. 41B10.13-FITC staining after stimulation with hCD40L plus hIL-4 for 3 days. FIG. 9D: Another dot plot of anti-hCD19-PE vs. isotype-FITC staining on day 0. FIG. 9E: A dot plot of anti-hCD19-PE vs. 59E6.8-FITC staining on day 0. FIG. 9F: A dot plot of anti-hCD19-PE vs. 59E6.8-FITC staining after stimulation with hCD40L plus hIL-4 for 3 days. The pattern of anti-hpen staining is essentially unchanged from day 0. Similar findings were obtained using mAb 29A6.2-FITC or after stimulation with PWM (20 μg/ml) plus hIL-4 (20 ng/ml)(not shown). Activation of B cells was confirmed by increased expression of hCD69 (not shown).

FIGS. 10A to 10C reveal that in vivo administered mAb 29A6.2 dramatically reduced the size of the spleen and the number and size of lymphoid follicles. Same-age, same-sex F1 littermates of B6×Balb/C crosses were given an intraperitoneal injection of Incomplete Freund's Adjuvant to induce ascites to facilitate hybridoma growth 5 days before intraperitoneal implantation of five million SP 2/0 AG14 (negative control; fusion partner) or hybridoma 29A6.2 cells. One week after hybridoma implantation, spleens were removed, weighed and fixed in formaldehyde for pathology examination. FIG. 10A is a photograph of the spleen of a mouse implanted with SP2/0 AG14 (weight: 169.7 mg)(left) compared with that of a mouse implanted with 29A6.2 (weight: 80.2 mg)(right). The ruler at top shows length in cm.

FIG. 10B shows a representative field of the spleen from the mouse implanted with SP2/0 AG14 shown in 10A. FIG. 10C shows a representative field of the spleen from the mouse implanted with hybridoma 29A6.2 shown in 10A. Note the decrease in the size and number of lymphoid follicles and the distorted architecture in C. Hematoxylin-eosin stain. All microphotographs are shown at the same magnification (40× original). One of three independent experiments with similar results.

FIGS. 11A and 11B are micrographs of live BaF3/mpen cells stained with the chimeric mouse-human IgG1 mAB mh29A6.2. Cell culture supernatants (300 microliter per 1×10⁵ target cells) of HEK293 cells stably transfected with mh29A6.2 mAb light- and heavy-chain expression vectors were incubated with BaF3/mpen. Cells were washed with H5FAH and bound antibodies were detected by FITC-anti-human IgG Fc. FIG. 11A is a phase-contrast micrograph. FIG. 11B is an immunofluorescence micrograph of the same cells shown in FIG. 11A. The staining pattern is identical to that of the parental 29A6.2 mAb. Bar indicates 200 micron. Cells stained with the supernatants of HEK293 cells transfected with empty (negative control) vectors showed no staining (not shown).

### DETAILED DESCRIPTION OF THE INVENTION

In the description that follows, a number of terms and conventions commonly used in immunology and molecular biology are utilized. In order to provide a clear and precise understanding of the specifications and claims, including the scope to be given such terms, the following definitions are provided.

Following the convention of protein and gene naming and used herein, “pen” refers to penumbra protein of any species. “mpen” refers to mouse penumbra protein specifically. “hpen” refers to human penumbra protein specifically. Pen is the abbreviation of the mouse Penumbra gene while PEN refers to human PENUMBRA gene. Tetraspanin 33 is the alternative name for penumbra in GenBank, abbreviated as Tspan33. As used herein, “protein”, “peptide”, “peptide fragment” and “partial fragment of protein or antibody” include polymers of three or more amino acids linked by consecutive peptide bonds. No distinction based on length is intended between a protein, a peptide, a peptide fragment or a partial fragment of protein (including antibody). The term “antibody” is art-recognized terminology and includes intact or active partial fragments of antibodies that bind to target antigens. The term immunoglobulin (Ig) may be used in place of antibody. Each heavy chain and each light chain of a naturally occurring antibody contains one “variable region” (V region or VR) located in the amino terminal portion of the peptide chain (VH or VL, for heavy or light chain variable regions, respectively). Each VR is about 110 amino acids long (excluding the N-terminal signal peptide) and contains regions of variability in amino acid sequences that distinguish the antibody made by one B (or plasma) cell clone from that of a different clone. The VR of one heavy chain is juxtaposed with the VR of one light chain for form an antigen-binding site. Most of the amino acid sequence differences among different antibodies are localized to three short segments in the VRs of heavy and light chains and termed the “hypervariable region”. Hypervariable regions are about ten amino acids long each, and they are held in place by more conserved amino acid sequences known as the “framework regions” (FR) of the VR. In each pair of disulfide-bonded heavy and light chains, the three hypervariable regions of a VL domain and the three hypervariable regions of a VH domain cluster together to form a three-dimensional antigen-binding surface. The hypervariable regions are also called “complementarity-determining regions” (CDRs) as they together create a three-dimensional surface that is complementary to the topology of the cognate antigen or epitope like a lock and its key. Fab stands for “fragment, antigen binding” and consists of a complete light chain coupled to a VH and the first constant region of the heavy chain. Therefore, Fab retains the ability to bind to its cognate antigen. Fabs that retain the heavy-chain hinge are called Fab′; when the interchain disulfide bonds are preserved, the two Fab′ fragments remain linked to produce a divalent form called F(ab′)₂. Both Fab′ and F(ab′)₂ can recognize its cognate antigen(s). In this application, the term “antibody” is used in its broadest sense and specifically covers, but is not limited to, monoclonal antibodies, polyclonal antibodies, multi-specific antibodies as well as their active partial fragments. Examples of active partial fragments of antibodies that bind to target antigens include, but not limited to, Fab, F(ab′)₂, “domain antibody fragment” (VR alone of a heavy or light chain) and “diabody” (ref. 33). The term “monoclonal antibody” (mAb) as used in the art refers to a preparation of homogeneous antibody molecules and is used to differentiate it from “polyclonal antibody”. A chimeric antibody refers to any antibody or partial antibody fragments composed of genetically engineered amino acid sequences derived from two or more antibodies from different animal species (or strains) or different classes or subclasses of Igs, so long as they exhibits the desired biological activities. A “humanized antibody” refers to an antibody created by transferring the immunoglobulin variable or hypervariable region (plus possibly some FR) amino acid sequences or corresponding cDNAs or genes of a nonhuman antibody into the corresponding regions of human immunoglobulin amino acid sequences or cDNAs or genes. Almost all murine mAbs can be humanized by grafting their CDRs onto the FR of human antibodies. A humanized antibody has the advantage of being less likely to trigger adverse immune responses when given to humans and will work more effectively with the immune system of the recipients. “Conservative amino acid substitution” refers to substitution of amino acids that, as known to those skilled in the art, may be made generally without altering the biological activity of the resultant molecule. Examples of conservative amino acid substitution include, but not limited to, substitution of Ala with Gly or Ser; Arg with Lys or His; Asn with Gln or His; Asp with Glu or Asn; Cys with Ser or Ala; Gln with Asn; Glu with Asp or Gln; Gly with Ala; His with Asn or Gln; Ile with Leu or Val; Leu with Ile or Val; Lys with Arg or His; Met with Leu or Ile or Tyr; Phe with Tyr or Met or Leu; Pro with Ala; Ser with Thr; Thr with Ser; Trp with Tyr or Phe; Tyr with Trp or Phe; Val with Ile or Leu. Antibodies can be covalently (cleavable or noncleavable in vivo) linked to drugs or other functional groups (e.g. drugs, ligands, receptors, binding proteins, enzymes, fluorochromes, radioisotopes, metal particles) to serve various purposes while exploiting the antigen-antibody specificity. They can also be modified covalently (e.g. PEGylation, glycosylation) or noncovalently (e.g. ionic interaction) to alter their behavior in vivo such as stability, volume of distribution, renal ultrafiltration, lipid solubility, blood-brain barrier crossing, metabolism and antigen processing by particular cell types or enzymes.

As used herein, “erythrocytes” refers to “red blood cells” (already enucleated) and “erythroblasts” refers to erythroid precursors that still contain nuclei and have not completed the entire terminal differentiation process. “Young erythrocytes” refers to new erythrocytes in blood circulation or spleen and have just been released from bone marrow microenvironment. They are similar the so-called “shift cells”. “MNCs” stands for “mononuclear cells”, which are obtained by density-gradient centrifugation fractionation of blood, marrow or spleen cell preparations. “ACK lysis” refers to removal of erythrocytes by lysis using a hypotonic buffer containing ammonium chloride and potassium bicarbonate.

The following MATERIALS AND METHODS were used in the examples that follow.
Establishment of cell lines stably expressing cell surface mpen or hpen.

The cloning of the cDNA of mouse Pen (mPen) and human PEN (hPEN) has been described by the present inventor (ref. 3). The construction of the pcDNA3.1/mpen vector has also been described (ref. 3). Briefly, the entire coding region of mpen was cloned into the HindIII-XhoI sites of the pcDNA3.1 expression vector (Invitrogen). The resultant vector expressed mpen as a fusion protein with a Myc-His_{×6} tag in the intracellular C-terminus. The vector was electroporated into the pen-negative mouse “pre-B” cell line BaF3 (American Type Culture Collection or ATCC; BaF3 does not express mpen) and transfected cells were selected with G418 (1 mg/ml) for 10 days as described (ref. 3). Stable transtectants, hitherto referred to as “BaF3/mpen”, was first established in 2004 and used in the current anti-pen mAb project. Stable BaF3/mpen were screened for expression of the fusion protein by immunofluorescence staining of methanol-fixed cytospin preparations using purified anti-Myc mAb conjugated with rhodamine (Santa Cruz Biotechnology), followed by fluorescent microscopy. Immunoprecipitation using an anti-Myc mAb (clone 9E10)(Santa Cruz Biotechnology) and anti-mpen C-terminus rabbit antisera demonstrated that the stable transfectants produced the 39-kilodalton full-length mpen fused to a Myc-Hisₓ₆ tag as predicted. As a negative control, BaF3 was transfected with the negative control vector pcDNA3.1 and selected with G418 (1 mg/ml) for 10 days to produce a stable line BaF3/pcDNA3.1. An hpen expression vector was constructed by ligating the entire coding sequence of hpen into the BgIII-HpaI sites of the retroviral expression vector MSCV-PGK-EGFP (ref. 34). The hpen protein was expressed from the long-terminal repeat (LTR)-enhancer of MSCV. The enhanced green fluorescent protein (EGFP) was expressed independently from the internal phosphoglycerate kinase (PGK) promoter. BaF3 was transfected with MSCV-hpen-PGK-EGFP (or the parental vector MSCV-PGK-EGFP as a negative control) and pSV2Neo (as a selectable marker) at 10:1 molar ratio using GenePulser Xcell (BioRad). Transfected cells were selected with G418 (1 mg/ml) for 10 days to obtain stable transfectants, hitherto referred to as BaF3/hpen. BaF3 and its derivatives were cultured in RPMI medium (Gibco) supplemented with fetal bovine serum (FBS; 10% vol/vol)(Hyclone), 2-mercaptoethanol (2-ME; 1×10⁻⁶ M)(Sigma) and WEHI 3B conditioned medium (10% vol/vol) as a source of mouse interleukin-3 (IL-3).

### Immunization, Fusion, Screening and Subcloning of Hybridomas.

Four- to six-weeks-old female C57Black/6 (“B6”) or Balb/C or Pen “knockout” (in-frame deletion of first and second TMs; ref. 3) mice were immunized intraperitoneally with 1×10⁷ BaF3/mpen (or BaF3/hpen) emulsified in complete Freund's adjuvant (CFA; MP Biomedicals) and boosted with 2-4×10⁶ BaF3/mpen (or BaF3/hpen) intravenously without adjuvant. Four days after the boost, spleens were harvested and single cell suspension was prepared by lysis with ammonium chloride lysis buffer (ACK, 017.2) and fused with SP2/0 AG14 myeloma cells (ATCC) using polyethylene glycol (Roche), plated in 96-well plates and selected with HAT (hypoxanthine, aminopterin and thymidine)(Sigma). Supernatants of hybridomas were screened by indirect immunofluorescence using BaF3/mpen as positive indicators and fluorescein isothiocyanate (FITC)-conjugated goat anti-mouse immunoglobulins (GAMIgs)(BD Pharmingen) as the secondary antibody. Positive supernatants were rescreened with BaF3 and BaF3/pcDNA3.1 to exclude non-pen-specific clones. Pen-specific clones were subcloned by limiting dilution at 0.5 cells/well, rescreened and subcloned again by limiting dilution at 0.2 cells/well. Wells were inspected after seeding to identify those with only one cell. Clonal lines were maintained in RPMI supplemented with 10% FBS, 4-(2-hydroxyethyl)-1-piperazineethanesulfonic acid (HEPES; 0.01 M, pH 7.2)(Fisher Biotech) and 2-ME (1×10⁻⁵ M). The class of antibody was determined using the ISOSTRIP™ mouse monoclonal antibody isotyping kit (Roche Diagnostics)

Purification and Conjugation of mAbs.

mAbs were purified from tissue culture supernatant using agarose-recombinant protein A (Abcam), which does not bind bovine serum albumin or bovine Igs but has variable binding capacity for mouse IgM. Purified antibodies were conjugated with FITC per manufacturer's instructions (Abcam).

Preparation of Hematopoietic Cells from Spleen, Bone Marrow, Lymph Node and Blood.

Spleen, bone marrow, lymph node and peripheral blood cells were obtained from Balb/C or B6 mice and treated with ACK lysis buffer to remove most erythrocytes. Alternatively, erythrocytes (as well as polymorphonuclear cells) were removed by density gradient centrifugation through a step-gradient of NYCOPREP™ (p=1.077 g/ml)(Nycomed). In some experiments, whole bone marrow was analyzed without ACK lysis or NYCOPREP™ density centrifugation. For human hematopoietic cells, heparinized blood was diluted with RPMI at a 1:5 ratio and centrifuged through a step-gradient of FICOLL-PAQUE™ Plus (p=1.077 g/ml)(Amersham Pharmacia Biotech). Light-density cells (ρ<1.077 g/ml) were collected and washed before staining with antibodies or culturing.

### Immunofluorescence Staining, Flow Cytometry and Fluorescence Microscopy.

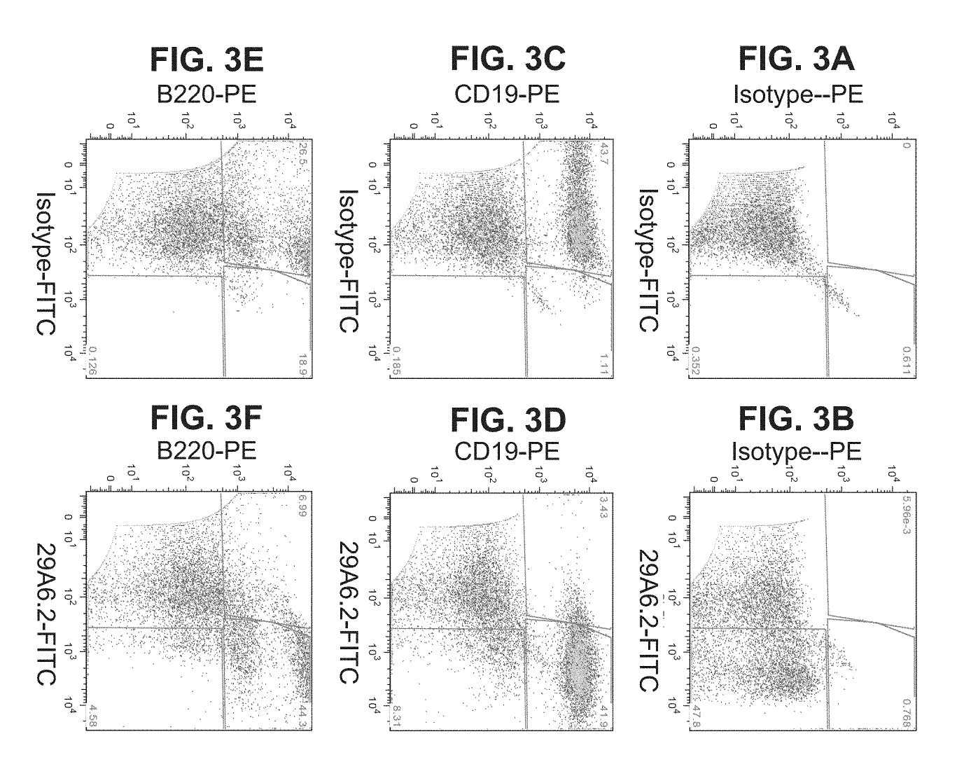

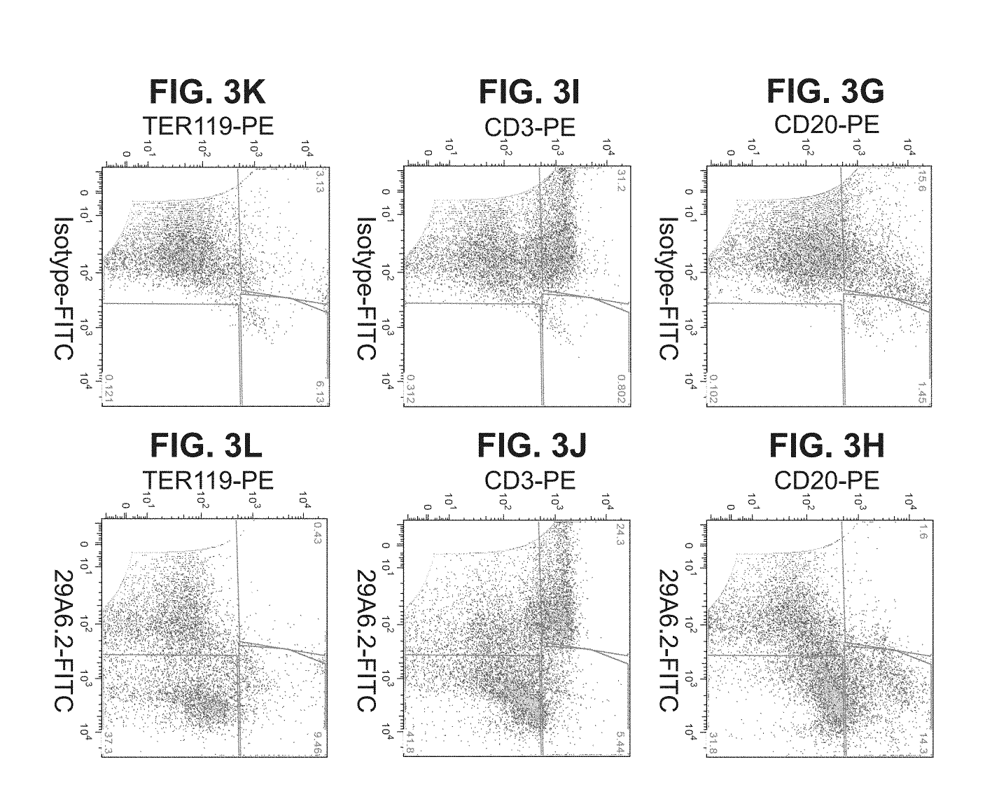

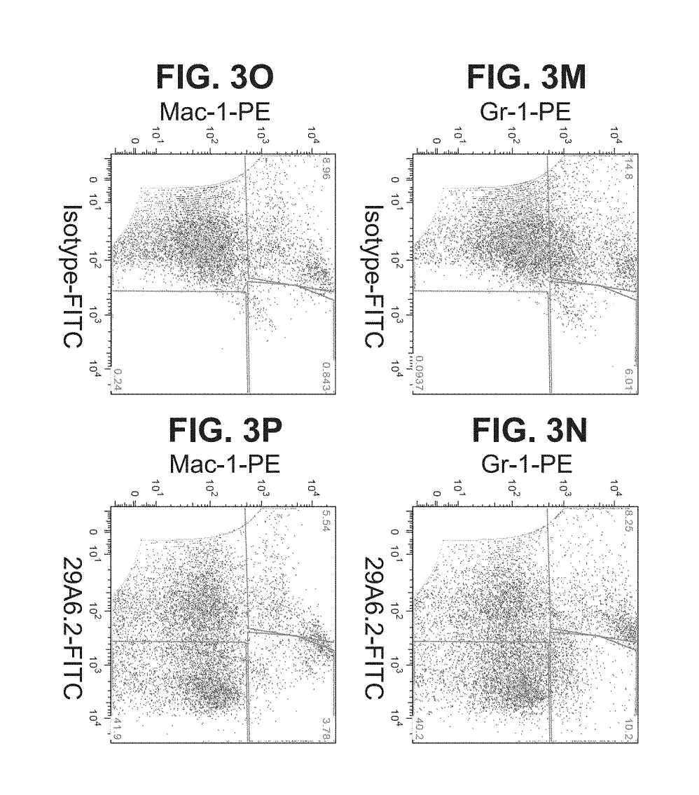

The medium used for cell staining was Hank's Balanced Salt Solution (HBSS) supplemented with heat-inactivated FBS (5% vol/vol) and sodium azide (0.09% wt/vol). This solution is known as “H5FAH”. All staining and washing were performed at 4° C. The flow cytometer used was a DxP flow cytometer with CYTOTEK™ upgrade (Cytotek). Analysis of flow cytometry data was performed using FLOWJO™ v. 9 (FIG. 3 only) or v.10 (Beckton-Dickinson)(BD). Fluorescence microscopy was performed using a microscope equipped with a digital camera. Anti-mouse antibodies used in this study include the following: (anti-)CD19-phycoerythrin (PE)(clone 6D5; eBioscience), CD20-PE (clone AISB12; Affymetrix), B220-PE (clone RA3-6B2; BioLegend), CD3e-PE (clone 145-2C11; eBioscience), CD4-PE (clone GK1.5; eBioscience), CD-8-PE (clone 53.6.7; eBioscience), Gr-1-PE (clone RB6-8C5; BioLegend), Mac-1a-PE (clone M1/70; Affymetrix), NK1.1-PE (clone PK136; Affymetrix), DX5-PE (clone DX5; BioLegend), TER119-PE (Clone TER-119; eBioscience), CD16/32 (FcBlock; anti-FCγIII/II receptor; BD), CD25-PE/Cy7 (clone PC61; BD Pharmingen), CD62L (clone MEL-14; BD Pharmingen), CD69-APC (clone H1.2F3; BioLegend), CD80-APC (clone 16-10A1; BioLegend), CD86-APC (clone GL-1; BioLegend), CD117-PE (anti-c-kit; clone ack45; eBioscience), CD117-APC (clone 2B8; eBioscience), CD138-PE (clone 281-2; BD), mouse IgG1 kappa isotype-PE (clone MOPC-21; BD), GAMIgs-PE (BD), GAMIgs-FITC (BD), GAMIgG1-PE (BD), GAMIgG2a-PE (BD), GAMIgG2b-PE (BD) and GAMIgM-PE or FITC (BD), anti-mouse IgM-PE/Cy7 (clone RMM-1; BioLegend), anti-mouse IgD-APC (clone 11-26c 2a; BioLegend) and matching isotype controls. Anti-human antibodies used in this study include the following: (anti-)CD19-PE (clone SJ25C1 and 4G7; BD), CD20-PE (clone 2H7; BioLegend), B220-PE (clone RA3-6B2; BioLegend), CD3-PE (clone HIT3a; BD), CD14-PE (clone HCD14; BioLegend), CD235a-PE (clone HI264; BioLegend), CD138-PE (clone DL-101; BioLegend), CD4-PE (clone OKT4; BioLegend), CD8-PE (clone SK1; BioLegend) and CD69-APC (clone FN50; BioLegend). For the immunofluorescence detection of chimeric mouse-human IgG1 anti-pen mAbs by microscopy or flow cytometry, a FITC-conjugated mAb against human IgG Fc domain was used (clone HP6017; BioLegend).

### Activation of B Cells In Vitro.

Four to six million light-density (ρ<1.077 g/ml) human peripheral blood mononuclear cells (MNC) were cultured in 2 ml of RPMI supplemented with 20% FBS, glutamine, nonessential amino acids, sodium pyruvate, HEPES and 2-ME (1×10⁻⁶ M), human IL4 (hIL-4; 20 ng/ml)(R&D Systems) without or with human IL-2 (hIL-2; 2 ng/ml)(Chiron) plus or minus human CD40 ligand (hCD40L; 200 ng/ml)(BioLegend) or PWM (20 μg/ml)(Sigma). Human B cells do not express Toll-Like Receptor 4 (TLR4) for LPS but can be activated by PWM. Mouse spleen MNCs were cultured in the same medium supplemented with hIL-4 (20 ng/ml) without or with hIL-2 (2 ng/ml) and PWM or E. coli LPS (20 ng/ml)(Sigma). Cells were analyzed after culturing for 24-72 hrs.

### In Vivo Effects of mAb 29A6.2.

All anti-pen hybridomas described in this application resulted from the fusion of the spleen cells of B6 mice with the myeloma cell line SP2/0 AG14 (Balb/C origin). Therefore F1 progenies from Balb/C×B6 crosses were used as the hosts in implantation studies to prevent rejection of hybridomas. To minimize variables, only female littermates were used in each study. Five to 10 days before implantation of hybridomas, each mouse was given an intraperitoneal injection of 0.1 ml of Incomplete Freund's Adjuvant (IFA)(MP Biomedicals) emulsified with phosphate buffered saline (PBS; pH7.2) to induce ascites to support hybridoma growth. Mice were then injected with 5×10⁶ hybridoma cells (either 29A6.2 or the parental line SP2/0 AG14) intraperitoneally. After 5-10 days mice were euthanized and spleens and kidneys were photographed and weighed. The kidneys and spleens were fixed in 3.7% formaldehyde in PBS and embedded in paraffin. Paraffin sections were stained with hematoxylin-eosin (HE stain)(Sigma).

Determination of the cDNA and Amino Acid Sequences of the Entire Heavy- and Light-Chain Variable Regions of Anti-Pen Hybridomas.

cDNA syntheses and rapid amplification of cDNA ends were performed using poly(A)+ mRNAs and primers located in the constant regions of mouse IgM heavy- and kappa light-chain constant region 1. The 5′ ends of cDNAs were dC-tailed using terminal deoxynucleotidyl transferase (BioLab). 5′ rapid amplification of cDNA ends (5′ RACE) was performed using nested constant region 1 (of p heavy chain or K light chain)-specific primers and 5′ RACE Abridged Anchor Primer (Life Technologies) that incorporated desired restriction sites in the 5′ or 3′ ends. PCR products were digested with appropriate restriction enzymes and cloned into the EcoRI/SalI (for light chains) or BamHI/SalI (for heavy chains) sites of pBluescript and plasmid DNAs (five or more clones per heavy- or light-chain) were prepared and Sanger sequencing was performed using T3 and T7 primers. Sequences were checked against all known databases using the BLAST program to verify their uniqueness and translatability. Signal peptides were identified using the SignalP-4.1 program (cbs.dtu.dk/services/SignalIP-4.1).

Creation of Chimeric Mouse-Human IgG1 Anti-Pen mAbs.

The entire coding sequences (including start codons and signal peptides) of the variable regions of the heavy- and light-chain cDNAs of hybridoma 29A6.2, 41B10.13 and 59E6.8 were amplified by PCR using Elongase (Invitrogen) from corresponding, full-length 5′ RACE cDNA clones in Bluescript and ligated in frame into the EcoRI-NheI sites of pFUSE-CHIg-hG1 vector (InvivoGen) to create chimeric heavy-chains consisting of mouse heavy-chain variable regions fused to the human IgG1 heavy-chain constant region, and into the BstEII/BsiWI sites of pFUSE2-CLIg-hk (InvivoGen) to create chimeric light-chains consisting of mouse light-chain variable regions fused to human kappa light-chain constant region. A Kozak consensus sequence (5′GCCGCCACC) was inserted immediately upstream of the start codon to Improve protein translation. The chimeric constructs were Sanger sequenced to ensure correct coding. Chimeric mouse-human heavy- and light-chain expression constructs were co-transfected into the HEK293 cell line and selected with Zeocin and/or Blasticidin and/or G418 (if pSV2Neo is co-transfected). Multiple clonal lines were established for each pair of chimeric heavy and light chains. The chimeric mouse-human mAbs were named according to the origin of the heavy and light chains with a prefix mh. Thus “mh29A6.2” refers to antibodies produced by HEK293 expressing the heavy- and light-chain variable regions of hybridoma 29A6.2 fused with human IgG1 heavy-chain and kappa light-chain constant domains, respectively. The entire antigen-binding site of 29A6.2 is reconstituted in “mh29A6.2” but the rest of antibody is derived from human IgG1/kappa light chain. To test the antigen-binding activity of chimeric antibodies, supernatants of individual HEK293 transfectants (clones) were incubated with BaF3/mpen or hpen that were pretreated with FcBlock. After washing, bound chimeric antibodies were detected by a FITC-conjugated mAb against human IgG Fc domain (clone HP6017; BioLegend). The supernatants of empty (control) vector-transfected HEK293 were used as negative controls.

### Example 1

### Establishment of Hybridomas 29A6.2, 41B10.13 and 59E6.8.

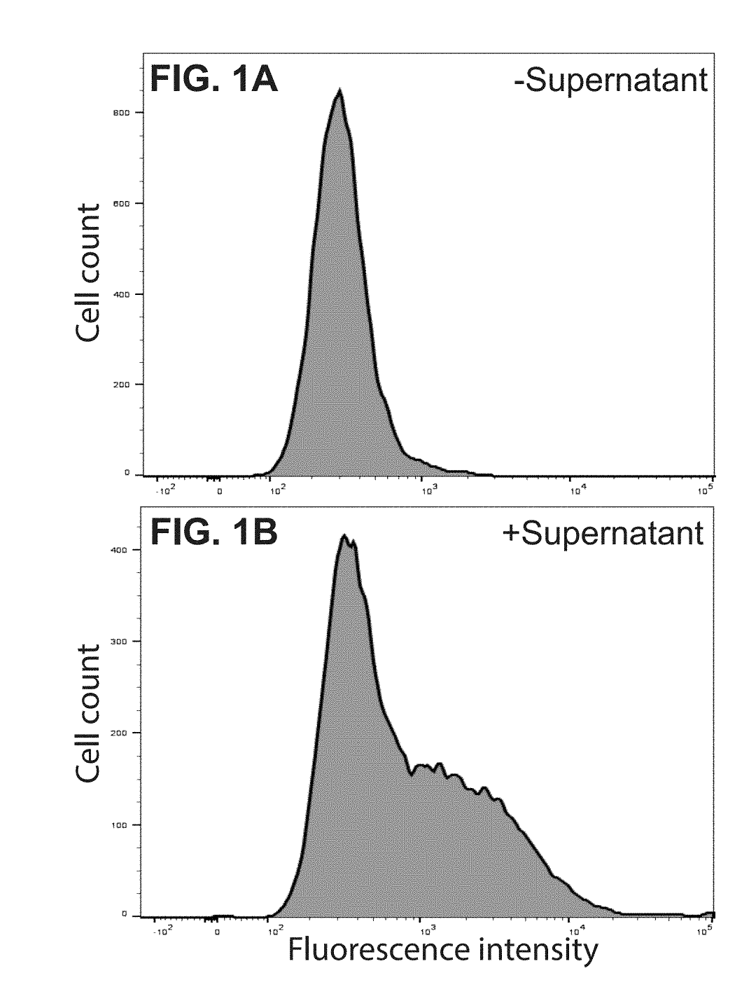

Spleen cells of immunized mice were fused with the myeloma cell line SP2/0 AG14 and selected with HAT for 10-14 days. Supernatants from all wells with hybridomas were screened by indirect immunofluorescence using BaF3/mpen as the positive indicator and BaF3 and BaF3/pcDNA3.1 as negative controls. Multiple clones fulfilling such criteria were subcloned by limiting dilution, expanded and further characterized. Three independent clones, 29A6.2, 41B10.13 and 59E6.8, are described in this application, although many more positive wells were identified in the primary screen. As shown in FIGS. 1A and 1B, hybridoma 29A6.2 produced an antibody that recognized the extracellular domain of mpen in live BaF3/mpen but not in the negative control cell line BaF3/pcDNA3.1 or any other mouse cell lines that do not express Pen mRNA by PCR or Northern analysis including NIH3T3, MPRO, WEHI 3B, BLL, EL4 and KIL (not shown).

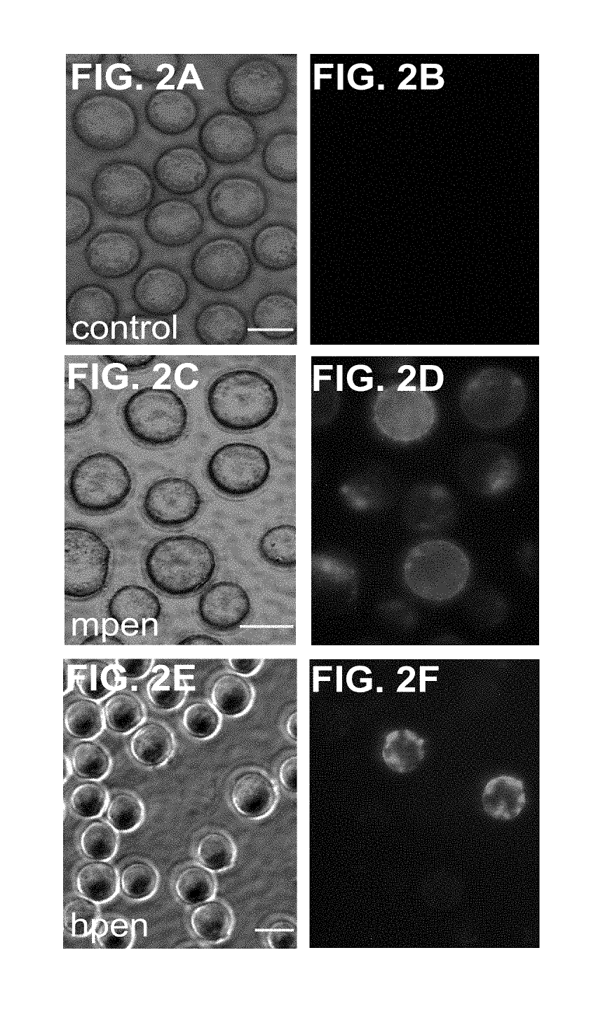

mAb 29A6.2 (or 41B10.13 or 59E6.8) recognized cell surface mpen in BaF3/mpen in immunofluorescence microscopy (FIGS. 2A to 2D) as well as hpen in BaF3 transfected with the MSCV-hpen-PGK-EGFP vector (FIGS. 2E&F). Isotyping showed that these three mAbs belong to the IgM class with kappa light chains.

### Example 2

### Detection of Penumbra-Expressing Cells in Normal Mouse Spleens.

mAbs 29A6.2, 41B10.13 and 59E6.8 were purified from tissue culture supernatant using agarose-recombinant protein A and conjugated with FITC. Mouse spleen cells were treated with ACK lysis buffer to remove RBC, pre-incubated with FcBlock and stained with FITC-conjugated mAb plus a PE-conjugated lineage-specific antibody and analyzed by flow cytometry. As shown in FIGS. 3C&D and G&H, almost all CD19-positive or CD20-positive mouse spleen mononuclear cells (MNCs) also expressed pen. B220 (Ly45R) is another commonly used B cell marker in mice. As shown in FIG. 3E, B220+ cells exhibited different levels of expression and could be roughly segregated into B220^{Hi} and B220^{Lo} populations. Both B220^{Hi} and B220^{Lo} populations expressed pen, with B220^{Hi} cells expressing slightly higher levels of pen (FIG. 3F). T cells (CD3+) and granulocytes (Gr-1+ or alternatively CD14+) did not express pen (FIGS. 3I&J and M&N). The majority of Mac-1-positive cells were negative for pen but a small fraction of Mac-1 (CD11c or αM integrin chain)+ cells had detectable levels of pen expression (FIGS. 3O&P). They were likely B lymphocytes expressing the αM integrin chain. NK cells (NK1.1+ in B6 mice; DX5+ in Balb/C mice) and platelets exhibited no detectable levels of pen (not shown). Interestingly, a small percentage of blood cells that survived ACK buffer lysis expressed low levels of pen as well as low levels of glycophorin A (recognized by the TER-119 mAb in mice)(FIGS. 3K&L). These TER119^{Lo} pen^{Lo} erythrocytes likely represented newly released (from bone marrow) erythrocytes that had not completed the terminal differentiation process. They are estimated to be less than 0.0001% of circulating erythrocytes. Mature erythrocytes expressed very high levels of glycophorin A (out of range of the plot in FIG. 3K) and no detectable pen.

In the initial study, all live cells (based on exclusion of the fluorescent dye 4′,6-diamidino-2-phenylindole or DAPI) were included in the flow cytometry analyses to establish the complete spectrum of hematopoietic cells that expressed cell surface pen. The analyses were repeated on live “lymphocyte”-gated cells only (as defined by forward and side scatters; also contains non-lymphocytes). The findings were essentially the same or very similar. Therefore, most analyses focused on live “lymphocyte”-gated populations unless stated otherwise.

### Example 3

Anti-Pen mAbs Recognize B Lymphocytes Regardless of their Activation Status.

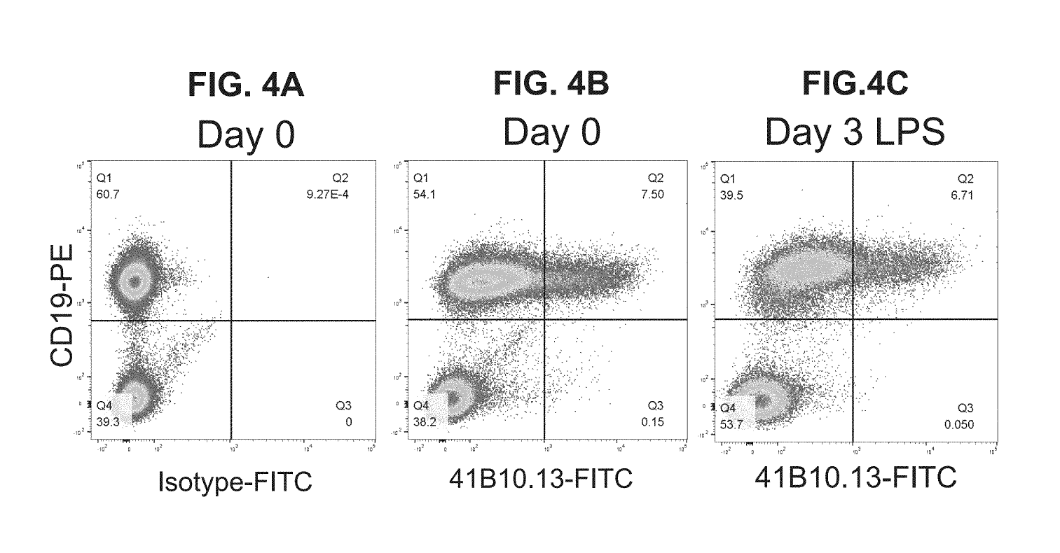

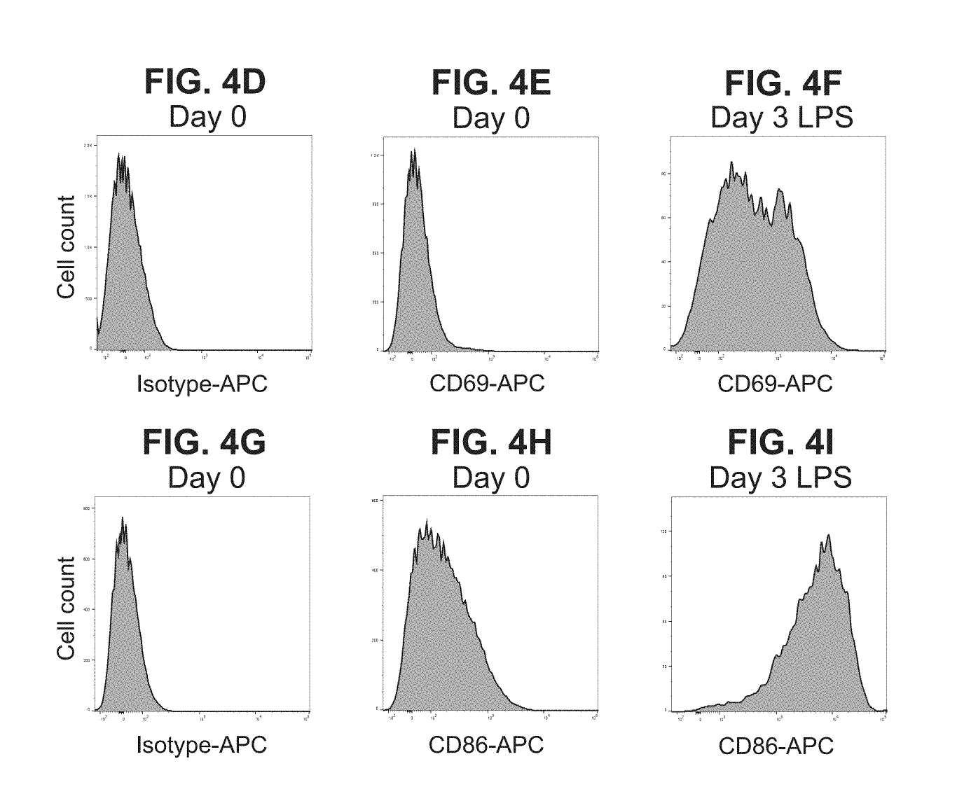

To study the effect of B lymphocyte activation on the expression of pen, mouse spleen MNCs were stimulated in vitro with IL-4 and E. coli LPS (with or without IL-2). Cells were harvested 24-72 hrs. after stimulation and stained with 29A6.2-FITC (or 41B10.13-FITC or 59E6.8) and CD19-PE and analyzed by flow cytometry. As shown in FIGS. 4A-C, there were little changes in anti-pen staining before (FIG. 4B) and after activation (FIG. 4C). However, there were dramatic increases in the expression of activation markers such as CD69 (FIGS. 4D-F), CD86 (FIGS. 4G-I) and CD80 (not shown) in CD19+ cells, confirming successful activation of B cells by LPS and IL-4. Similar results were obtained after PWM stimulation (not shown). These findings indicate that anti-pen mAbs 29A6.2, 41B10.13 and 59E6.8 recognized both non-activated and activated B cells in spleen and the expression of cell surface pen in B cells was not significantly affected by activation.

### Example 4

### Expression of Penumbra in Normal Mouse Bone Marrow Cells.

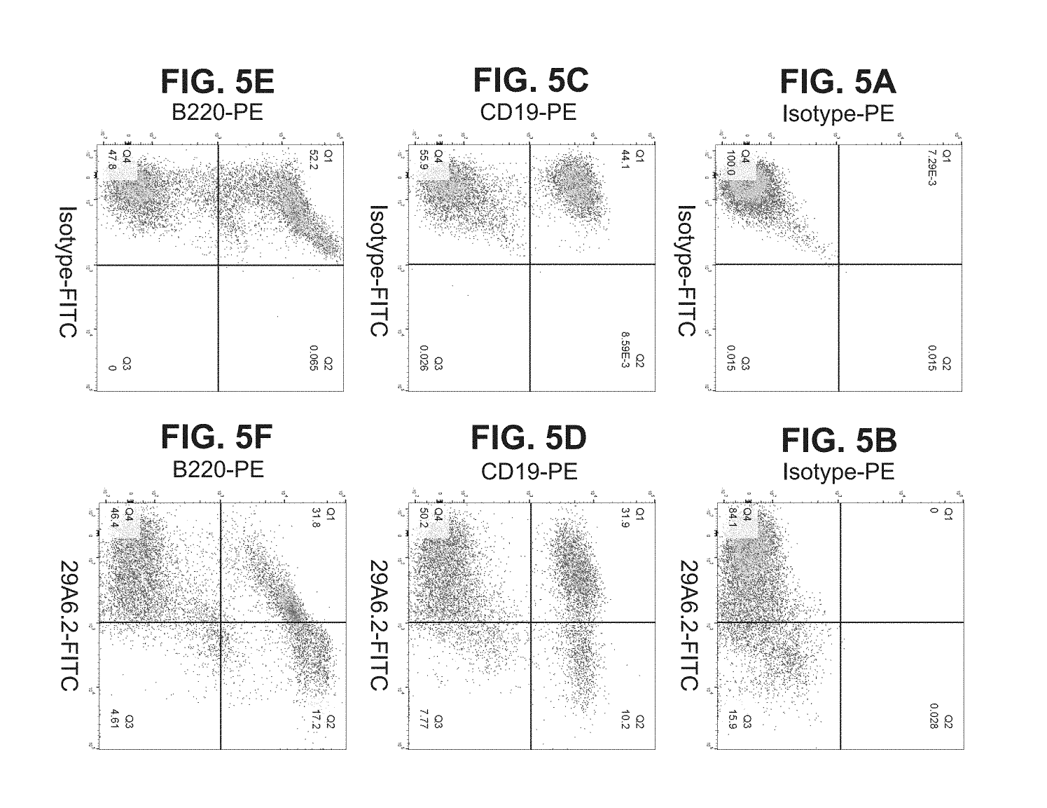

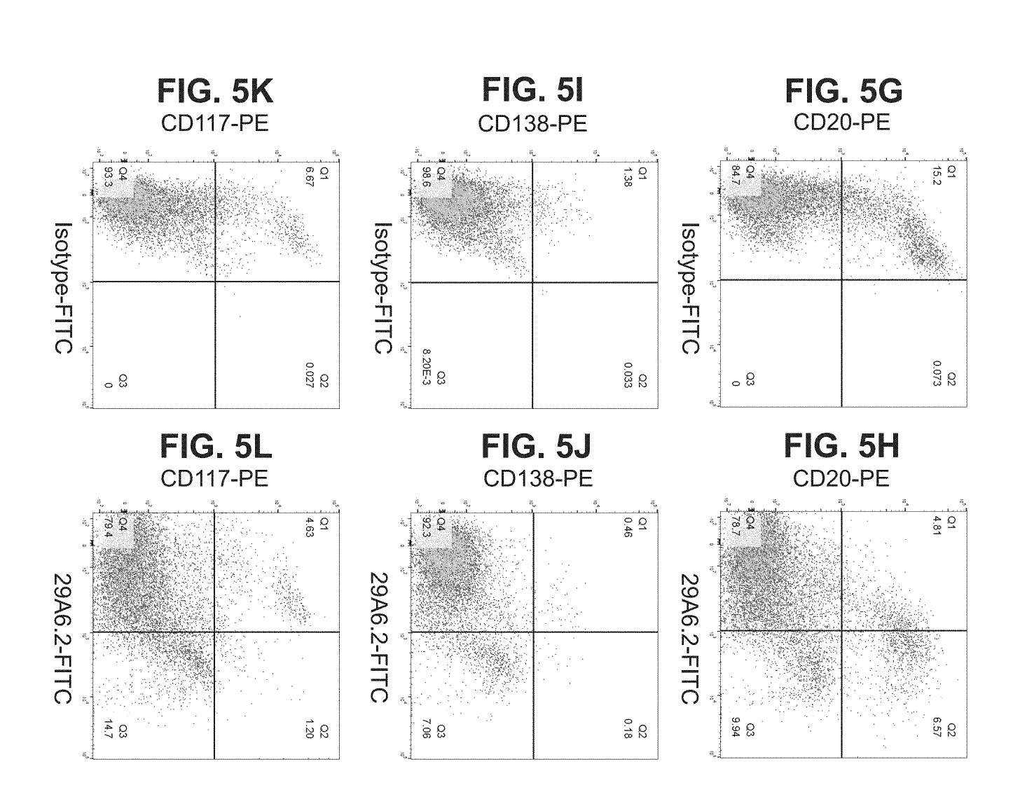

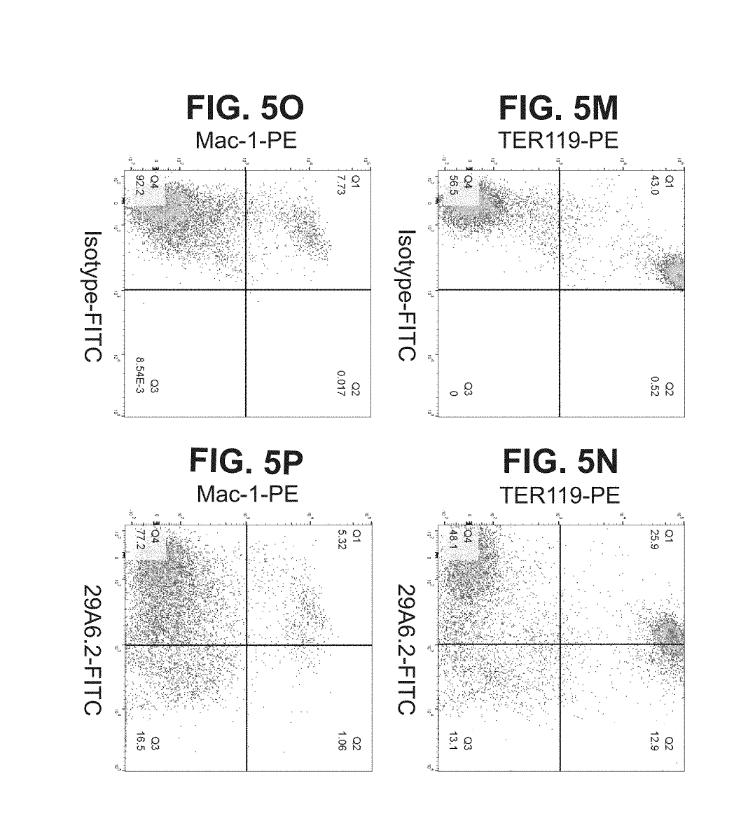

The results of flow cytometry analyses of mouse bone marrow cells (after lysis of most erythrocytes by ACK buffer) were similar to those of spleen cells except that a higher fraction of CD19+ B cells expressed lower levels of pen (FIGS. 5c&n) and the percentage of B220+ cells (FIGS. 5E&F) was higher than that in spleen (FIGS. 3E&F), consistent with bone marrow's role as the primary site of B cell development. In addition, the frequency of TER119+ erythrocytes and/or erythroblasts (FIGS. 5M&N) was higher than that in spleen (FIGS. 3K&L), consistent with the bone marrow's role as the main site of erythropoiesis. Furthermore, a very small percentage of immature hematopoietic progenitors expressing c-kit (CD117; stem cell factor or SCF receptor) also expressed low levels of pen (FIGS. 5K&L). These c-kit^{Lo} pen^{Lo} bone marrow cells were likely differentiating erythroblasts, some of which are known to express c-kit. Interestingly, most CD138 (syndecan-1)+ cells (plasmablasts and plasma cells) were negative for pen but a small fraction of CD138+ cells expressed low levels of pen (FIGS. 5I&J). The rare pen+CD138+ cells most likely represented plasmablasts based on their low frequency. Taken together, the results of bone marrow flow cytometry indicated that mAb 29A6.2 (or 41B10.13 or 59E6.8) recognized most CD19+ or CD20+ B cells in the bone marrow as well as a very small population of glycophorin A^{Lo} erythrocytes and/or erythroblasts.

### Example 5

### Expression of Penumbra in Normal Mouse Blood Cells.

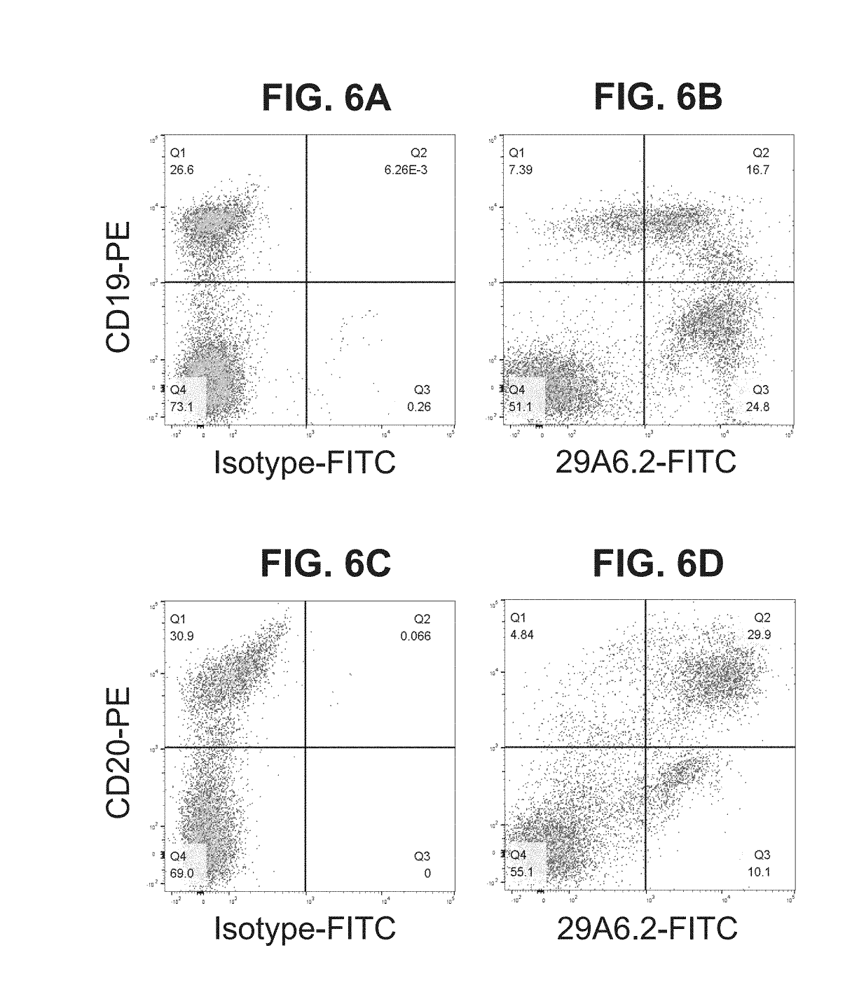

Mouse blood cells were lysed by ACK buffer and treated with FcBlock and stained with various antibodies. As shown in FIGS. 6A&B, virtually all circulating CD19+ B cells expressed some cell surface pen. Similarly, the great majority of CD20+ B cells also expressed pen (FIGS. 6C&D). Co-staining with non-B lineage-specific mAbs (anti-CD3, -CD14, -Gr-1, -Mac-1, -NK1.1 or DX5) was essentially negative (not shown). The results were consistent with the findings in spleen and bone marrow describe above.

### Example 6

### Expression of Pen in B Lymphocytes in Normal Mouse Lymph Nodes.

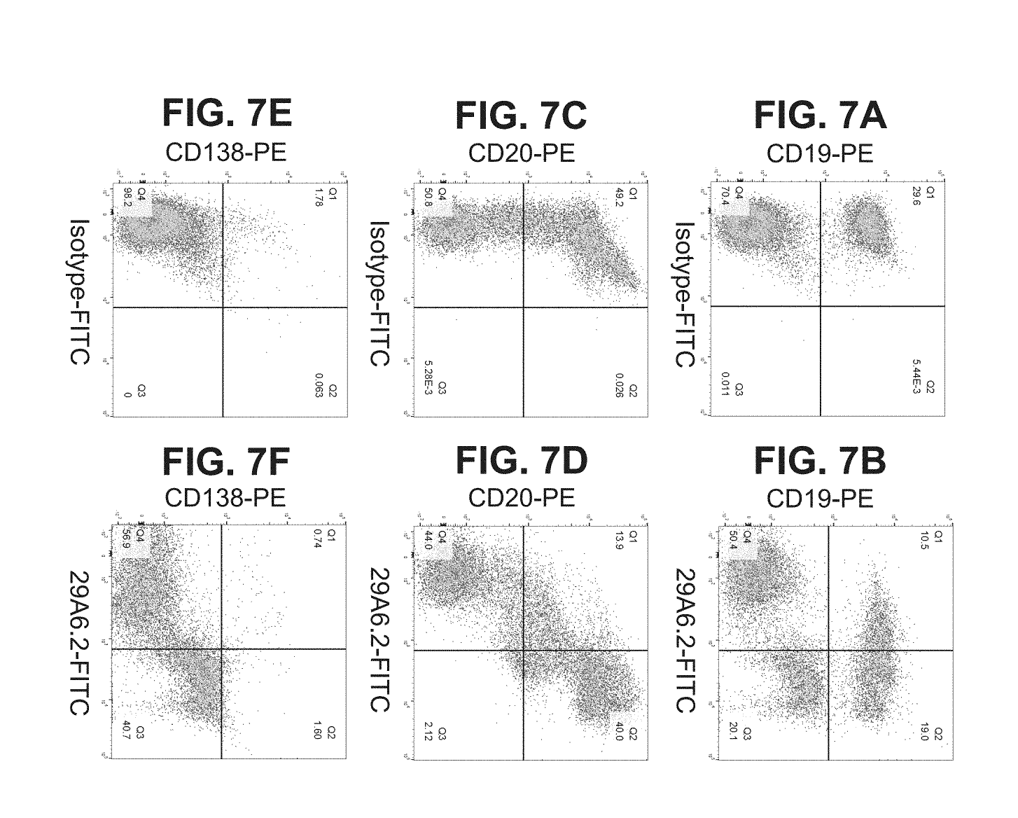

Single cell preparations of omental and cervical lymph nodes were subjected to ACK buffer lysis, treated with FcBlock and stained with FITC-conjugated anti-pen mAbs and PE-conjugated lineage-specific antibodies. The results again showed that most of the CD19+ or CD20+ lymph node B lymphocytes also expressed pen (FIGS. 7A to 7D). Again, the CD138+ lymph node cells were largely negative for pen expression (FIGS. 7E and 7F).

### Example 7

mAbs 29A6.2, 41B10.13 and 59E6.8 Recognize Human CD19+ B Cells in Blood.

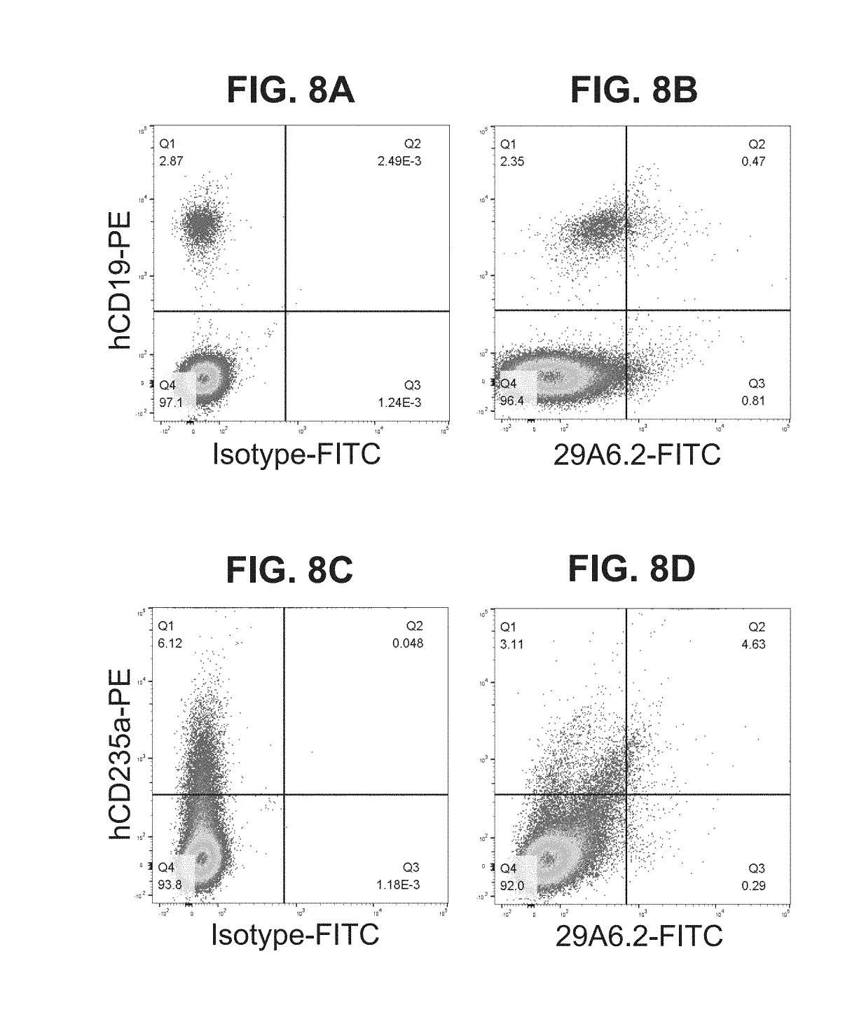

As illustrated in FIG. 2, mAbs 29A6.2, 41B10.13 and 59E6.8 also recognized cell-surface hpen in cells transfected with a hpen expression construct. To determine if mAb 29A6.2 (or 41B10.13 or 59E6.8) also recognized hpen on circulating human B cells, mononuclear cells were obtained from heparinized human blood by Ficoll-Hypague density centrifugation and stained with FITC-conjugated 29A6.2 (or 41B10.13 or 59E6.8) plus CD19-PE or other lineage specific antibodies. As shown in FIGS. 8A and B, most circulating human CD19+ B cells were recognized by 29A6.2, 41B10.13 or 59E6.8, although 59E6.8 had slightly weaker staining. The majority of circulating human CD19+ B cells expressed low-to-medium levels of hpen with a smaller subpopulation (about 2.5%) expressing higher levels of hpen. Most circulating human CD20+ B cells also express hpen (not shown). mAbs 29A6.2, 41B10.13 and 59E6.8 did not recognize circulating human CD3+T lymphocytes, CD14+ leukocytes (neutrophil and monocyte) or mature RBC (not shown). As in the mice, mAbs 29A6.2, 41B10.13 and 59E6.8 recognized a small population of glycophorin^{Lo} erythrocytes in peripheral blood (FIGS. 8C and D). Thus the ability of mAbs 29A6.2, 41B10.13 and 59E6.8 to recognize cell surface pen and the expression pattern of pen in blood cells are similar In human and mouse.

### Example 8

mAbs 29A6.2, 41B10.13 and 59E6.8 Recognize Human B Cells Regardless of their Activation Status.

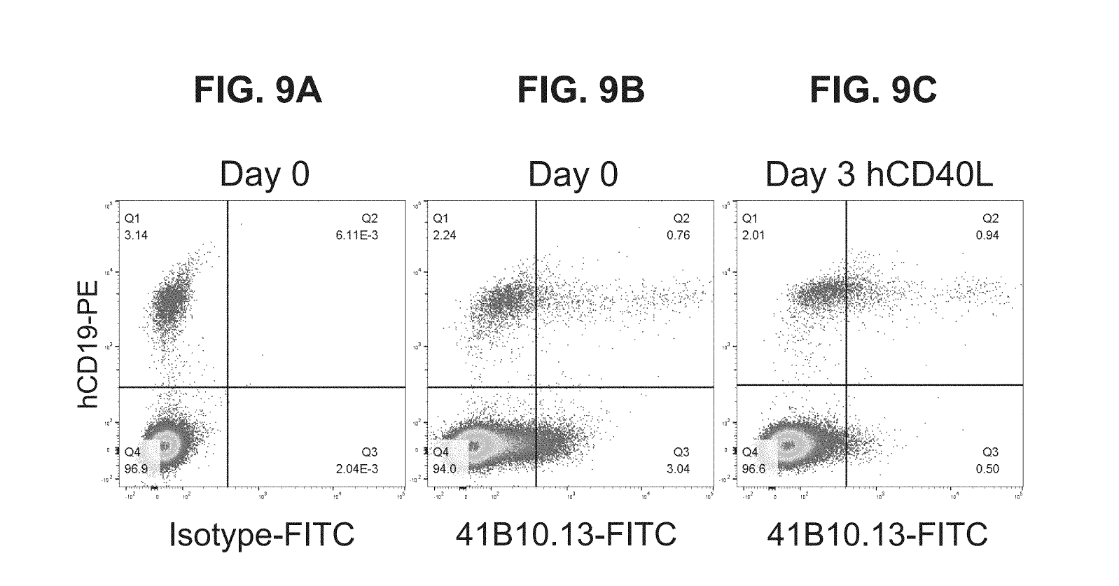

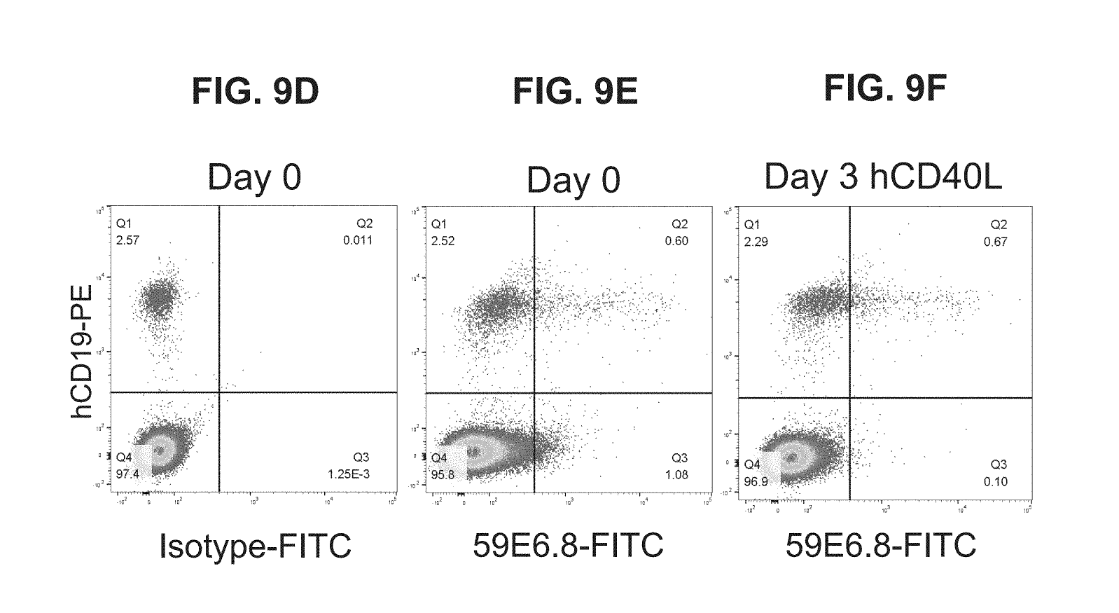

Unlike mouse B cells, human B cells do not express the toll-like receptor 4 (TLR4; the receptor for LPS) and therefore cannot be activated by LPS. However, human B cells can be activated in vitro by hCD40L plus hIL-4 or PWM plus hIL-4. Therefore we compared the staining of human B cells with anti-hCD19-PE plus 41B10-FITC before and after stimulation with hCD40L plus hIL-4 for 3 days. As shown in FIG. 9, there was no significant difference in anti-pen staining before and after activation. Similar results were obtained after stimulation with PWM plus hIL-4 or substitution of 41B10.13-FITC with 29A6.2-FITC (not shown) or 59E6.8-FITC (FIGS. 9D to F). Thus anti-mpen mAbs can recognize hpen in non-activated as well as activated human B lymphocytes and the expression of hpen in normal human blood B cells did not increase with their activation. This corroborated the findings in mouse splenic B cells described above (FIG. 4).

### Example 9

### Anti-Pen mAb Greatly Reduces the Size of Spleen In Vivo.

The ability of anti-mpen mAbs to recognize both human and mouse cell-surface pen and the conserved expression patterns in both species made it possible to extrapolate or predict the biological effects of anti-pen mAbs in humans by examining their effects in mice. To study the possible effects of mAb 29A6.2 on mouse splenic B cells, we implanted the hybridoma 29A6.2 cells intraperitoneally in F1 progenies of B6×Balb/C crosses. As hybridoma 29A6.2 shares the genetic background of Balb/C (SP2/0 AG14) and B6 (fusion partner) mice, the hybridoma cells or their secreted immunoglobulins would not be rejected by the B6×Balb/C F1 progenies. To support the growth of the implanted hybridomas, the recipient F1 mice were injected with incomplete Freund's adjuvant (IFA) to induce ascites 5 days prior to implantation of hybridomas or SP2/0 AG14 (negative control). After 7-10 days, spleens were harvested, weighed, photographed, fixed in formaldehyde and the sections examined microscopically.

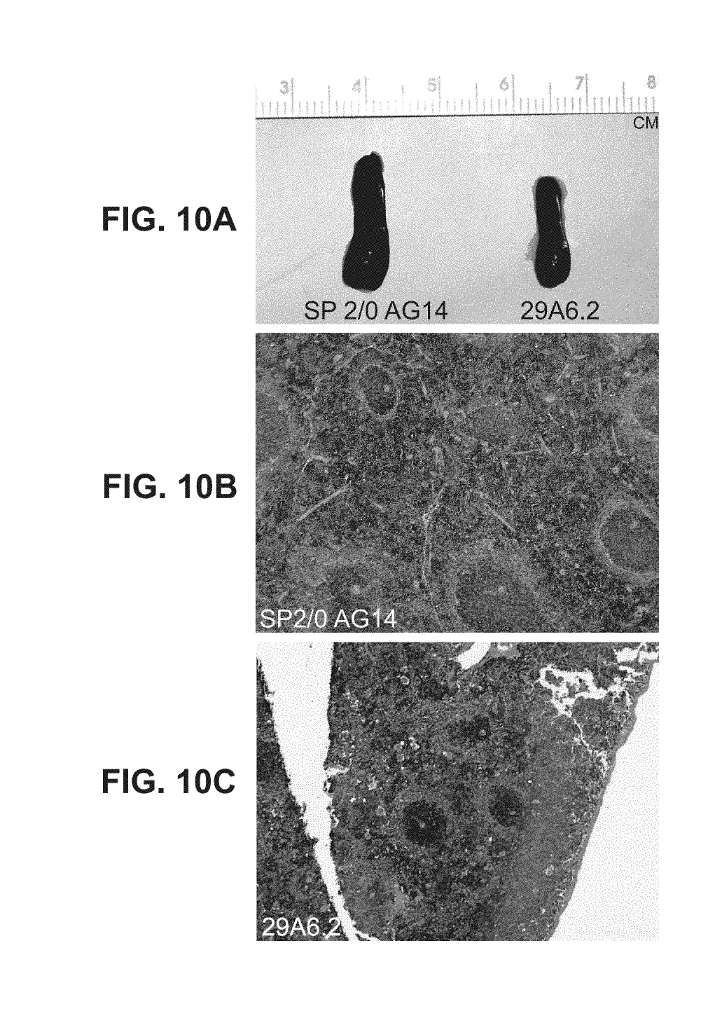

As shown in FIG. 10A, the spleens of mice implanted with the 29A6.2 hybridoma exhibited several abnormalities: (1) The spleens were decreased by about 50% in terms of size and weight compared with mice implanted with SP2/0 AG14 (negative control); (2) The spleens of mice implanted with 29A6.2 were macerated compared with those implanted with SP2/0 AG14, indicating destruction of the splenic tissue; (3) Pathological examination revealed greatly reduced numbers and size of lymphoid follicles and more chaotic architecture (probably due to the destruction of lymphoid follicles) in spleens of mice implanted with hybridoma 29A6.2 (FIGS. 10B and 10C). Taken together, these findings indicate that binding of mAb 29A6.2 to B cells led to their destruction in vivo.

### Example 10

Determination of the Nucleotide and Amino Acid Sequences of the Entire Variable Regions of mAbs 29A6.2, 41B10.3 and 59E6.8.

Complementary DNAs (cDNAs) of the entire VRs of heavy and light chains were obtained by 5′ RACE and cloned into pBluescript and Sanger sequenced. These cDNAs contain the 5′ untranslated regions, the entire coding sequences of the VRs including the start codons, the signal peptides and the first parts of the constant regions. The complete amino acid sequences of the VRs (including signal peptides) of the heavy- and light-chains of mAbs 29A6.2, 41B10.13 and 59E6.8 were deduced from the cDNA sequences. The signal peptides of all chains were identified using SignalP-4.1 software.

SEQ ID NO:1-3 correspond to the entire VR amino acids sequences (including signal peptides) of the p heavy chains of mAbs 29A6.2 (SEQ ID NO:1), 41B10.13 (SEQ ID NO:2) and 59E6.8 (SEQ ID NO:3). In all three, the first 19 amino acids represent the signal peptides that are removed by signal peptidase in the mature p heavy chains.

SEQ ID NO:4 corresponds to the entire VR amino acid sequences (including signal peptide) of the K light chains of mAbs 29A6.2, 41B10.13 and 59E6.8. These three mAbs share the same K light chain but their heavy chains are different. Amino acids 1-19 of SEQ ID NO:4 represent the signal peptide of the K light chain that is removed by signal peptidase in the mature K light chain.

### Example 11

Creation of Chimeric Mouse-Human IgG1 Chimeric mAbs by Recombinant DNA Technology.

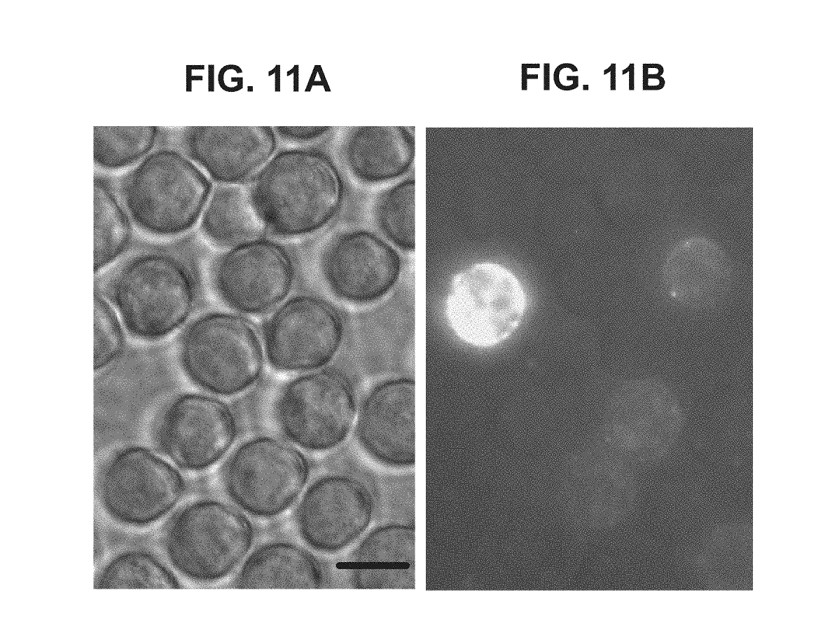

Chimeric mouse-human IgG1 mAbs were constructed as detailed in MATERIALS AND METHODS. FIG. 11B illustrates the specific staining of BaF3/mpen cells by the chimeric “mh29A6.2” mAb. It is identical to the staining pattern of the parental 29A6.2 mAb (for comparison, please see FIGS. 2D&2F). Furthermore, the titers of the chimeric anti-pen mAbs in the supernatants of clonal HEK293/mh29A6.2 were similar to that of the original 29A6.2 hybridoma.

### CONCLUSION, RAMIFICATIONS AND SCOPE

This invention describes the creation and characterization of anti-mpen mAbs and the demonstration that anti-mpen mAbs described in this application recognize human pen (hpen) equally well. Furthermore, these mAbs recognize the native pen protein displayed on living human and mouse cells. Importantly, these mAbs recognize virtually all human and mouse CD19+ or CD20+ or B220+ B lymphocytes and do so regardless of their activation status. In addition, we have determined the nucleotide and amino acid sequences of the entire VRs of the heavy and light chains of these mAbs including their signal peptides. Finally, using recombinant DNA technology, we created chimeric mouse-human IgG1 mAbs consisting of the entire VRs of the mouse mAbs grafted in frame onto the constant regions of human IgG1 heavy chain and kappa light chain.

The creation of anti-pen mAbs followed many years of research, starting with the cloning of the human and mouse Penumbra genes (ref. 1-3), followed by the elucidation of their domain structures (ref. 3) and more recently, the identification of their membrane protein partners (ADAM-10) and their possible role in the proteolytic activation of the Notch receptor (upon binding of Notch ligands)(ref. 4-6) and possibly other signaling events. These early efforts provided important clues as to how best to immunize and screen for antibodies that could recognize native pen on living cells. However, the cloning of the human and mouse Penumbra genes was the crucial event.

### ADVANTAGES

Although anti-pen mAbs recognize virtually all CD19+ or CD20+ B lymphocytes, they exhibit a very different staining profile than anti-CD19 or anti-CD20 mAbs. While staining with fluorochrome-conjugated anti-CD19 mAbs resulted in relatively uniform fluorescence intensity in the CD19+ population (usually within one log or a 10-fold range)(FIGS. 3C, 4A, 5C, 6A, 7A, 9A), staining of the same population of cells using fluorochrome-conjugated anti-pen mAbs yielded fluorescence intensity that ranged over 3 to 4 logs (1,000 to 10,000-fold difference), with the majority of the positive cells expressing low to intermediate levels (FIGS. 3D, 4B, 5D, 6B, 7B, 9B)(Flow cytometry data in FIG. 3D was analyzed using FloJo v.9, which provided the best demonstration of the wide range of expression level particularly at the lower end). This wide range of expression level of pen among human and mouse B lymphocytes suggests that cell-surface level of pen may be determined by factors such as the stages of B cell development or further differentiation (e.g. affinity maturation of Ig, isotype switching, memory) of mature B cells or microenvironment or cell cycling or cytokine modulation or concentrations of co-factors (e.g. ADAM10) or shuttling between cell surface and internal membrane compartments or other physiological processes. This wider range of pen cell-surface expression level provides an opportunity to select or target subpopulations of B lymphocytes with different levels of cell-surface pen. For example, it may be possible to differentially target malignant or abnormal B cells (e.g. diffuse large B-cell lymphoma, chronic lymphocytic leukemia, acute lymphocytic leukemia, Hodgkin's lymphoma, Burkitt lymphoma, autoimmune B cells, etc.) with higher levels of cell-surface hpen and spare B cells expressing lower levels of hpen. This may reduce complications such as B cell aplasia and hypoglobulinemia that are often seen in anti-CD20 therapy.

Another advantage of the anti-pen mAbs described in this disclosure is that since the same mAbs can recognize both mouse and human pens, it is possible to predict some of the behavior and side effects of these mAbs (or the chimeric human-mouse versions) in humans by studying the existing mAbs in mice. This may save a lot of time, effort and expense in preclinical studies of anti-pen mAbs.

A significant advantage of the chimeric mouse-human IgG1 anti-pen mAbs described in this application is that that the human IgG1 Fc portion can bind to protein A or G or a number of IgG1 Fc-binding proteins more efficiently. This will make facilitate the purification of these chimeric mAbs. Another advantage is that the human IgG1 Fc does not bind to mouse FcγIIIR. Thus, the chimeric mouse-human mAbs described in this application may be used to study mouse blood cells without pretreatment of mouse cells with FcBlock. The chimeric antibodies also have advantages over IgM mAbs in applications that benefit from the smaller size of the chimeric IgG1 antibodies. Importantly, human IgG1 has a stronger potential for antibody-dependent cell-mediated cytotoxicity (ADCC) in humans, which translates into more effective killing of target cells. Finally, the 293 HEK cells are of human origin. Therefore, the chimeric mAbs purified from these producer cells are less likely to contain impurities that may trigger adverse immune reactions when administered into humans.

The anti-pen mAbs described in this application provide useful tools in the study of normal B cell development and later stages of erythropoiesis. They may also find applications in the detection, diagnosis, immunophenotyping, classification and prognostication of lymphoid, erythroid and other malignancies in which the malignant cells express penumbra. Such malignancies may include, but not limited to, diffuse large B cell lymphoma (the most common B cell lymphoma), follicular B cell lymphoma, Hodgkin's lymphoma, Burkitt lymphoma, chronic lymphocytic leukemia, acute B lymphocytic leukemia and acute erythroleukemia. Anti-pen mAbs may also be used in combination with other therapeutic modalities such as radiation, surgery, chemotherapy, immunotherapy and biologic response modifiers. Example, anti-pen mAbs may be used together or sequentially with anti-CD20 immunotherapy to increase killing of lymphoma cells while reducing tumor resistance due to reductions in CD20 expression as a result of prior treatment, modulation or mutations. Many autoimmune disorders are caused or accompanied by autoimmune B cells such as systemic lupus erythematosus, rheumatoid arthritis, scleroderma, autoimmune hemolytic anemia and idiopathic thrombocytopenia. Thus, anti-pen mAbs may be used to treat autoimmune diseases characterized by disregulated B lymphocytes. About two thirds of transplant patients with an allogeneic bone marrow or hematopoietic progenitor transplant suffer from chronic graft-versus-host disease and many eventually die from it. Chronic graft-versus-host disease is in part caused by “autoimmune” (i.e. from the perspective of the transplant recipients) allogeneic B lymphocytes received or produced from the bone marrow or hematopoietic progenitor grafts. Thus, anti-pen mAbs may find applications in the treatment of chronic graft-versus-host disease as well. Additional usage may include the treatment of B lymphocytes infected with viruses such as Epstein-Barr virus (or cytomegalovirus) as in infectious mononucleosis and post-transplant lymphoproliferative disorder.

While the identification of very low numbers of pen^{Lo} erythrocytes in blood and/or erythroblasts in bone marrow confirmed our original description of pen mRNA expression in erythroblasts (ref. 3), the detection of this small population may warrant some theoretical consideration in light of the possible application of anti-pen mAbs in the eradication of B cell malignancies such as diffuse large B cell lymphoma, Hodgkin's disease and B cell leukemias as well as B cell-mediated autoimmunity. Although only a very small fraction (estimated to be less than 0.0001%) of erythrocytes and some bone marrow erythroblasts express low levels of pen, it may still be important to reduce the number of such pen^{Lo} erythrocytes and/or erythroblasts in therapeutic applications of chimeric or humanized anti-hpen mAbs in order to minimize or prevent side effects of hemolysis such as renal toxicity due to the release of free hemoglobin. In practice, most cancer patients already suffer from anemia caused by cancer per se or chemotherapy or both and therefore have rather inactive erythropoiesis with few newly released erythrocytes as reflected in very low reticulocyte counts and the need for red blood cell transfusions. In theory, erythropoiesis can be completely suppressed in such patients by transfusion of red blood cells to a relative high hemoglobin level (e.g. 12-15 g/dL; normal adult males have a hemoglobin level of 14-15 g/dL on average at sea level). High levels of hemoglobin suppress the secretion of erythropoietin, which in turn suppresses erythropoiesis. Thus, we believe that there are safe and effective ways to further reduce the already low number of pen^{t}′ erythrocytes should their presence pose any danger to patients receiving Immunotherapy with anti-pen mAbs. In addition, the side effects of hemolysis, if any, can be ameliorated by other measures such as (i) hyperhydration with or without diuretics to encourage a high urine output to reduce the toxicity of free hemoglobin on renal tissues, and (ii) alkalization of urine by intravenous infusion of sodium bicarbonate to prevent precipitation of free hemoglobin in renal tubules. Such measures have already been incorporated into current lymphoma or leukemia chemotherapy to prevent the so-called “tumor lysis syndrome” or in the management of potential transfusion reactions (hemolysis due to ABO or Rh blood type mismatch or anti-donor RBC antibodies). Thus, there already exist several effective measures in the current practice of hematology/oncology clinics to prevent or minimize possible side effects resulting from the interaction between anti-pen mAbs and a small number of pen^{Lo} erythrocytes. In fact, anti-pen mAbs may be used to treat erythroleukemias that express pen.

Although the description above provides important examples of embodiments, they should not be construed as limiting the scope of the embodiments of this invention but only as illustrations of several embodiments. For example, the chimeric mouse-human anti-pen mAbs may incorporate the constant regions of any human Ig class (IgG, IgM, IgA, IgD, IgE) or subclass (IgG1, IgG2a, IgG2b, IgG3, IgG4, IgA1, IgA2, etc.) or light chain (kappa, lambda). Also, they may be constructed using DNA or amino acid sequences of Igs derived from any species other than mouse and human such as baboon, horse, cow, sheep, camel, llama, dog, cat, rabbit, etc. Furthermore, they are not limited to monovalent, bivalent, multivalent or same-valent antibodies. A single peptide chain of any length or a combination of different peptide chains that retain the antigen-recognition capability of the anti-pen mAbs can be prepared and tested by those skilled in the art. In a further embodiment, the hypervariable-region (or CDR) amino acid sequences of the VRs of anti-pen mAbs can be identified using the IgBLAST software (ncbi.nlm.nih.gov) and grafted onto the FRs of human antibodies to create new mAbs with the same antigen specificity. Such “humanized” antibodies preserve the ability to recognize pen but have a lower potential for triggering allergic reactions in humans. The mAbs disclosed here can also be modified using the well-established method of “conservative amino acid substitution” by those skilled in the art to create mAbs with variant amino acid sequences without significantly impairing the essential pen-binding capacity. In general, alteration of the amino acid sequences outside the CDRs, i.e. in FRs and constant regions, may be made without abolishing the antigen-binding capacity. In addition, any of these molecules can be modified by covalent and noncovalent modifications to alter or impart new properties such as stability (e.g. PEGylation), solubility, body distribution, metabolism, antigenicity, cytotoxicity (e.g. with cytotoxic drugs, predrugs, inhibitors, radioisotopes, complement activators), signaling (e.g. with cytokines, ligands, receptors, enzyme inhibitors or activators), tracking (e.g. with fluorochromes, dyes, enzymes, binding proteins, gold particles, magnetic beads, radiopaque molecules, radioisotopes), etc. using available technology.

### REFERENCES

1. Tsai, S. June 9, 2000. GenBank accession no. AF276890 (mouse Penumbra cDNA and amino acid sequences) and AF276891 (human PENUMBRA cDNA and amino acid sequences).
2. Chen, Z., Pasquini, M., Hong, B., Dehart, S., Heikens, M., and Tsai, S. 2005. The human Penumbra gene is mapped to a region on chromosome 7 frequently deleted in myeloid malignancies. Cancer Genet Cytogenet. 162:95-98.
3. Heikens, M. J., Cao, T. M., Morita, C., Dehart, S. L., and Tsai, S. 2007. Penumbra encodes a novel tetraspanin that is highly expressed in erythroid progenitors and promotes effective erythropoiesis. Blood. 109:3244-3252.
4. Dunn, C. D., Sulis, M. L., Ferrando, A. A. and Greenwald, I. 2010. A conserved tetraspanin subfamily promotes Notch signaling in Caenorhabditis elegans and in human cells. Proc Natl Acad Sci USA. 107:5907-5912.
5. Haining, E. J., Yang, J., Bailey, R. L., Khan, K., Collier, R., Tsai, S., Watson, S. P., Frampton, J., Garcia, P., and Tomlinson, M. G. 2012. The TspanC8 subgroup of tetraspanins interacts with A disintegrin and metalloprotease 10 (ADAM10) and regulates its maturation and cell surface expression. J Biol Chem. 287:39753-39765.
6. Dornier, E., Coumailleau, F., Ottavi, J-F., Moretti, J., Boucheix, C., Mauduit, P., Schweisguth, F., and Rubinstein, E. 2012. TspanC8 tetraspanins regulate ADAM10/Kuzbanian trafficking and promote Notch activation in flies and mammals. JCB. 199: 481-496.
7. Oren, R., Takahashi, S., Doss, C., Levy, R., and Levy, S. 1990. TAPA-1, the target of an antiproliferative antibody, defines a new family of transmembrane proteins. Mol Cell Biol. 10:4007-4015.
8. Boucheix, C., and Rubinstein, E. Tetraspanins. 2001. Cell Mol Life Sci. 58:1189-1205.
9. Tarrant, J. M., Robb, L., van Spriel, A. B., and Wright, M. D. 2003. Tetraspanins: molecular organizers of the leukocyte surface. Trends Immunol. 24:610-617.
10. Stipp, C. S., Kolesnikova, T. V., and Hemler, M. E. 2003. Functional domains in tetraspanin proteins. Trends Biochem Sci. 28:106-112.
11. Hemler, M. E. 2005. Tetraspanin functions and associated microdomains. Nat Rev Mol Cell Biol. 6:801-811.
12. Yáñez-Mó, M., Barreiro, O., Gordon-Alonso, M., Sala-Valdés, M., and Sánchez-Madrid, F. 2009. Tetraspanin-enriched microdomains: a functional unit in cell plasma membranes. Trends Cell Biol. 19:434-446.
13. Charrin, S., Le Naour, F., Silvie, O., Milhiet, P. E., Boucheix, C., and Rubinstein, E. 2009. Lateral organization of membrane proteins: tetraspanins spin their web. Biochem J. 420:133-154.
14. Maecker, H. T., Todd, S. C., and Levy, S. 1997. The tetraspanin superfamily: molecular facilitators. FASEB J. 11:428-442.
15. Bradbury L E, Kansas G S, Levy S, Evans R L, and Tedder T F. 1992. The CD19/CD21 signal transducing complex of human B lymphocytes includes the target of antiproliferative antibody-1 and Leu-13 molecules. J Immunol. 149:2841-2850.
16. Matsumoto, A. K., Martin, D. R., Carter, R. H., Klickstein, L. B., Ahearn, J., and Fearson, D. T. 1993. Functional dissection of the CD21/CD19/TAPA-1/Leu13 complex of B lymphocytes. J Exp Med. 178:1407-1417.
17. Fearon, D. T., and Carter, R. H. 1995. The CD19/CR2/TAPA-1 complex of B lymphocytes: linking natural to ac-quired immunity. Annu Rev Immunol. 13: 127-149.
18. Horváth, G., Serru, V., Clay, D., Billard, M., Boucheix, C., and Rubinstein, E. 1998. CD19 Is Linked to the Integrin-associated Tetraspans CD9, CD81, and CD82. J Biol Chem. 273:30537-30543.
19. Levy, S., Todd, S. C., and Maecker, H. T. 1998. CD81 (TAPA-1): a molecule involved in signal transduction and cell adhesion in the immune system. Annu Rev Immunol. 16:89-109
20. Cherukuri, A., Shoham, T., Sohn, H. W., Levy, S., Brooks, S., Carter, R., and Pierce, S. K. 2004. The tetraspanin CD81 is necessary for partitioning of coligated CD19/CD21-B cell antigen receptor complexes into signaling-active lipid rafts. J Immunol. 172:370-380.
21. Mannion, B. A., Berditchevski, F., Kraeft, S., Chen, L. B., and Hemler, M. E. 1996. Transmembrane-4 Superfamily Proteins CD81 (TAPA-1), CD82, CD63, and CD53 Specifically Associate with Integrin α4β1 (CD49d/CD29). J Immunol. 157, 2039-2047.
22. Serru, V., Le Naour, F., Billard, M., Azorsa, D. O., Lanza, F., Boucheix, C., and Rubinstein, E. 1999. Selective tetraspan-integrin complexes (CD81/α4β1, CD151/α3β1, CD151/α6β1) under conditions disrupting tetraspan interactions. Biochem J. 340:103-111.
23. Doyle, E. L., Ridger, V., Ferraro, F., Turmaine, M., Saftig, P., and Cutler, D. F. 2011. CD63 is an essential cofactor to leukocyte recruitment by endothelial P-selectin. Blood. 118:4265-4273.
24. Barreiro, O., Zamai, M., Yanez-Mo, M., Tejera, E., Lopez-Romero, P., Monk, P. N., Gratten, E., Caiolfa, V. R., and Sanchez-Madrid, F. 2008. Endothelial adhesion receptors are recruited to adherent leukocytes by inclusion in preformed tetraspanin nanoplatforms. J Cell Biol. 183:527-542.
25. Stashenko, P., Nadler, L. M., Hardy, R., and Schlossman, S. F. 1980. Characterization of a human B lymphocyte-specific antigen. J Immunol. 125:1678-1685.
26. Tedder, T. F., Streuli, M., Schlossman, S. F., and Saito, H. 1988. Isolation and structure of a cDNA encoding the B1 (CD20) cell-surface antigen of human B lymphocytes. Proc Natl Acad Sci USA. 85:208-212.
27. Pescovitz, M. D. 2006. Rituximab, an anti-CD20 monoclonal antibody: history and mechanism of action. Am J Transplant. 6:859-866.
28. Huhn, D., Von Schilling, C., Wilhelm, M., Ho, A. D., Hallek, M., Kuse, R., Knauf, W., Riedel, U., Hinke, A., Srock, S., Serke, S., Peschel, C., and Emmerich, B. 2001. Rituximab therapy of patients with B-cell chronic lymphocytic leukemia. Blood. 98:1326-1331.
29. Gurcan, H. M., Keskin, D. B., Stern, J. N. H., Nitzberg, M. A., Shekhani, H., and Ahmed, A. R. 2009. A review of the current use of rituximab in autoimmune diseases. Internet Immunopharmacol. 9:10-25.
30. Prox, J., Willenbrock, M., Weber, S., Lehmann, T., Schmidt-Arras, D., Schwanbeck, R., Saftig, P., and Schwake, M. 2012. Tetraspanin15 regulates cellular trafficking and activity of the ectodomain sheddase ADAM10. Cell Mol Life Sci. 69:2919-2932.
31. Shoham, T., Rajapaksa, R., Boucheix, C., Rubinstein, E., Poe, J. C., Tedder, T. F., and Levy, S. 2003. The tetraspanin CD81 regulates the expression of CD19 during B cell development in a postendoplasmic reticulum compartment. J Immunol. 171:4062-4072.
32. Luu, V. P., Hevezi, P., Vences-Catalan, F., Maravillas-Montero, J. L., White, C. A., Casali, P., Llorente, L., Jakez-Ocampo, J., Lima, G., Vilches-Cisneros, N., Flores-Gutierrez, L, and Zlotnik, A. 2013. TSPAN33 is a novel marker of activated and malignant B cells. Clin Immunol. 149:388-399.
33. Hollinger, P., Prospero, T., and Winter, G. 1993. “Diabodies”: Small bivalent and bispecific antibody fragments. Proc Natl Acad Sci USA. 90:6444-6448.
34. DeKoter, R. P. and Singh, H. 2000. Regulation of B lymphocyte and macrophage development by graded expression of PU.1. Science. 288:1439-1441.

What is claimed is:

1. Monoclonal antibodies recognizing both human and mouse penumbra proteins.

2. Said monoclonal antibodies capable of recognizing penumbra protein on human and mouse B-lymphocytes.

3. Said monoclonal antibodies capable of recognizing penumbra protein on human and mouse B-lymphocytes regardless of their activation status.

4. Said monoclonal antibodies capable of recognizing penumbra protein on human and mouse B-lymphocytes and glycophorin A-low erythrocytes.

5. Monoclonal antibodies recognizing human penumbra protein.

6. The monoclonal antibodies of claim 5 capable of recognizing penumbra protein on human B lymphocytes.

7. The monoclonal antibodies of claim 5 capable of recognizing penumbra protein on human B lymphocytes regardless of their activation status.

8. Said monoclonal antibodies of claim 5 capable of recognizing penumbra protein on human B-lymphocytes and glycophorin A-low erythrocytes.

9. Monoclonal antibodies recognizing mouse penumbra protein.

10. The monoclonal antibodies of claim 9 capable of recognizing penumbra protein on mouse B lymphocytes.

11. The monoclonal antibodies of claim 9 capable of recognizing penumbra protein on mouse B lymphocytes regardless of their activation status.

12. The monoclonal antibodies of claim 9 capable of recognizing penumbra protein on mouse B lymphocytes and glycophorin A-low erythrocytes.

13. An anti-penumbra antibody and its producer cell line selected from the group consisting of:
(i) monoclonal antibodies 29A6.2, 41B10.13 and 59E6.8,
(ii) chimeric mouse-human IgG1 monoclonal antibodies mh29A6.2, mh41B10.13 and mh59E6.8,
(iii) a chimeric antibody or active partial fragment of a chimeric antibody that incorporates:
a. a heavy chain variable region comprising an amino acid sequence selected from SEQ ID NOs: 1, 2 and 3; or
b. a heavy chain variable region comprising an amino acid sequence selected from SEQ ID NOs: 1, 2 and 3 and conservatively modified amino acid substitutions in framework regions; or
c. a heavy chain variable region comprising an amino acid sequence selected from SEQ ID NOs: 1, 2 and 3 and up to three conservatively modified amino acid substitutions in complementarity determining regions; and
d. a light chain variable region comprising the amino acid sequence of SEQ ID NO:4; or
e. a light chain variable region comprising the amino acid sequence of SEQ ID NO:4 and conservatively modified amino acid substitutions in framework regions; or
f. a light chain variable region comprising the amino acid sequence of SEQ ID NO:4 and up to three conservatively modified amino acid substitutions in complementarity determining regions.

14. The antibody or active partial fragment of antibody of any of the claims 1 to 13, wherein the amino acid sequences are humanized.

15. A conjugate of antibody or active partial fragment of antibody of any of claims 1 through 14 with drugs, cytotoxic drugs, receptors, ligands, signal transducers, binding proteins, enzymes, another antibody, active partial fragment of another antibody, radioisotopes, fluorochromes, tracing molecules, metal particles or a functional group.

16. Utilization of antibody or active partial fragment of antibody of any of claims 1 through 15 in the detection, classification, diagnosis or treatment of human diseases including, but not limited to, erythroleukemia, B cell malignancies, B cell autoimmunity and B cell infections.


SEQUENCE LISTING (4 sequences)

SEQ ID NO: 1 - protein, 130 aa, Mus musculus
  MISC_FEATURE: 29A6.2 heavy chain variable region
  SIGNAL (1)..(19)
MAVLGLLFCLVTFPSCVLSQVQLKQSGPGLVQPSQSLSITCTVSGFSLTSYGVHWVRQSPGKGLEWLGVIWSGGSTDYNAAFISRLSISKDNSKSQVFFKMNSLQADDTAIYYCATFDYWGQGTTLTVSS

SEQ ID NO: 2 - protein, 141 aa, Mus musculus
  MISC_FEATURE: 41B10.13 heavy chain variable region
  SIGNAL (1)..(19)
MAVLALLLCLVTFPSCALSQVQLKESGPGLVAPSQSLSITCTVSGFSLTSYAISWVRQPPGKGLEWLGVIWTGGGTNYNSALKSRLSISKDNSKSQVFLKMNSLQTDDTARYYCAREGNAYYSNYESTAYWGQGTLVTVSA

SEQ ID NO: 3 - protein, 137 aa, Mus musculus
  MISC_FEATURE: 59E6.8 heavy chain variable region
  SIGNAL (1)..(19)
MGWSCIILILVAAATGVHSQVQLQQPGAELVKPGASVKMSCKASGYTFTSYWITWVKQRPGQGLEWIGDIYPGSGSTNYNEKFKSKATLTVDTSSSTAYMQLSSLTSEDSAVYYCARDYYGSSYDYWGQGTTLTVSS

SEQ ID NO: 4 - protein, 134 aa, Mus musculus
  MISC_FEATURE: 41B10.13 light chain variable region
  SIGNAL (1)..(19)
MRCSLQFLGVLMFWISGVSGDIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGSGTKLEIKRA